SEQUENCE LISTING (11 sequences)

SEQ ID NO: 1 - protein, 8 aa, Artificial Sequence
  Description of Artificial Sequence Synthetic peptide
LXXXXXXV

SEQ ID NO: 2 - protein, 8 aa, Artificial Sequence
  Description of Artificial Sequence Synthetic peptide
RXXXXXXX

SEQ ID NO: 3 - protein, 9 aa, Artificial Sequence
  Description of Artificial Sequence Synthetic peptide
XXXXXXXXX

SEQ ID NO: 4 - protein, 8 aa, Artificial Sequence
  Description of Artificial Sequence Synthetic peptide
RXXXXXXL

SEQ ID NO: 5 - protein, 8 aa, Artificial Sequence
  Description of Artificial Sequence Synthetic peptide
XXXXXXXX

SEQ ID NO: 6 - protein, 8 aa, Artificial Sequence
  Description of Artificial Sequence Synthetic peptide
XXXXXXXK

SEQ ID NO: 7 - protein, 8 aa, Artificial Sequence
  Description of Artificial Sequence Synthetic peptide
EXXXXXXX

SEQ ID NO: 8 - protein, 8 aa, Artificial Sequence
  Description of Artificial Sequence Synthetic peptide
XPXXXXXX

SEQ ID NO: 9 - protein, 7 aa, Artificial Sequence
  Description of Artificial Sequence Synthetic peptide
XXXXXXX

SEQ ID NO: 10 - protein, 8 aa, Artificial Sequence
  Description of Artificial Sequence Synthetic peptide
YLKDQQLL

SEQ ID NO: 11 - protein, 8 aa, Artificial Sequence
  Description of Artificial Sequence Synthetic peptide
XXKXXXXL

(19) United States
(12) Patent Application Publication
(10) Pub. No.: US 2005/0202458 A1
(43) Pub. Date: Sep. 15, 2005

(54) Antigen-specific cells, methods of generating these cells and uses thereof

(76) Inventors: Bruce L. Roberts, Southboro, MA (US); Charles A. Nicolette, Framingham, MA (US)

Correspondence Address: Antoinette F. Konski;Bingham McCutchen LLP, Suite 1800, Three Embarcadero Center, San Francisco, CA, 94111-4067, US

(21) Appl. No.: 10/940,113

(22) Filed: Sep. 13, 2004

Related U.S. Application Data
(63) Continuation of application No. 09/701,849, filed on Aug. 9, 2000, now abandoned.
(60) Provisional application No. 60/080,036, filed on Mar. 31, 1998.

(51) Int. Cl. C12Q 1/68; C12P 19/34

(52) U.S. Cl. 435/6; 435/91.2

(57) ABSTRACT

The present invention provides methods for identifying antigens recognized by immune effector cells. In one embodiment, the methods combine identifying amino acid sequence motifs of such antigens and the DNA sequences that potentially encode the identified amino acid motifs and the DNA sequences which are aberrantly expressed in the cells. By comparison of these sequences, novel antigens that are recognized by immune effector cells are identified.

### CROSS-REFERENCE TO RELATED APPLICATIONS

This application claims priority under 35 U.S.C. § 119(e) of provisional application Nos. 60/080,036, filed Mar. 31, 1998 and (Atty Docket No. 15979-30015.00, converted from U.S. Ser. No. 09/013,683) filed Jan. 26, 1998, the contents of which are hereby incorporated by reference into the present disclosure.

### TECHNICAL FIELD

This invention is in the field of molecular immunology and medicine. In particular, methods of identifying novel antigens recognized by immune effector cells and the genes encoding these antigens.

### BACKGROUND

In spite of numerous advances in medical research, cancer remains the second leading cause of death in the United States. In the industrialized nations, roughly one in five persons will die of cancer. Traditional modes of clinical care, such as surgical resection, radiotherapy and chemotherapy, have a significant failure rate, especially for solid tumors. Failure occurs either because the initial tumor: is unresponsive, or because of recurrence due to regrowth at the original site and/or metastases.

Cellular immunotherapy is emerging as a technologically and intellectually compelling anti-cancer treatment. The generation of an immune response against tumors has been demonstrated in several animal models and has been inferred from reports of spontaneous tumor regression in man (Stotter and Lotze (1990) Cancer Cells 2:44-55). Cytotoxic T-lymphocyte (CTL) responses can be directed against antigens specifically presented by tumor cells, both in vivo and in vitro, without the need for prior knowledge of the molecular mechanism by which the tumor arose. In animal models, established tumors can be eradicated by the adoptive transfer of T-cells that are specifically immune to the malignant cells (Beun et al. (1994) Immunol. Today 15:11-15). Techniques of adoptive T-cell therapy have recently been applied to the treatment of human viral disease, but the application of similar T-cell therapy for human malignancy has been hindered in part by the lack of well defined tumor antigens recognizable by autologous T-cells. Many human progressive or metastatic cancers, such as disseminated malignant melanoma or metastatic renal cell carcinoma, are resistant to conventional therapies, including chemotherapy and radiotherapy. In these types of cancers, immunotherapy has been tried over the past 10 years and although its success rate has been relatively modest, it remains a promising alternative to the conventional therapies (Bergmann et al. (1990) Onkologie 13:137).

In humans, spontaneous destruction of melanoma cells occurs in 15% to 20% of primary lesions, indicating that host protective mechanisms which can selectively destroy melanoma cells are present (Bystryn et al. (1993) Heme. Onc. Annals. 1:301). Vaccine immunotherapy with crude or partially purified melanoma vaccines can prevent tumor growth in 50% to 100% of mice immunized to otherwise lethal doses of melanoma cells. The protection is specific, indicating it is mediated by immune mechanisms. The challenge is to devise vaccine strategies that will induce similar immunoprotective responses in man.

In patients with cancer, specific CTLs have been often derived from lymphocytic infiltrates present at the tumor site (Weidmann et al. (1994) Cancer Immunol. Immunother. 39:1-14). These tumor infiltrating lymphocytes (TILs) are a unique cell population that can be traced back to sites of disease when they are labeled with indium and adoptively transferred.

Indeed, the presence of a large number of T-cells in tumors has been correlated with a prognostically favorable outcome in some cases (Whiteside and Parmiani (1994) Cancer Immunol. Immunother. 39:15-21). It has been shown that implantation of polyurethane sponges containing irradiated tumor cells can efficiently trap anti-tumor CTLs (4-times greater than lymph fluid, 50-times greater than spleen or peripheral blood) (Woolley et al. (1995) Immunology 84: 55-63). Following activation with T-cell cytokines in the presence of their appropriately presented recognition antigen, TILs proliferate in culture and acquire potent anti-tumor cytolytic properties (Weidmarin et al. (1994) supra). Thus, TILs are a convenient source of lymphocytes greatly enriched for cells with tumor cell specificity. Additionally, tumor-specific CTLs have been found in peripheral blood or malignant ascites of patients with cancer, indicating that a systemic response to the tumor may be present or that redistribution of CTLs from the tumor to the periphery might occur (Wallace et al. (1993) Cancer Res. 53:2358-2367). In either case, this is an attractive feature for the immunotherapeutic treatment of metastatic or disseminated cancers.

While isolation, expansion, and retransfusion of TILs is appealing, there are severe adverse cardiorespiratory and hemodynamic effects such as tachycardia, increases in cardiac index, systemic vascular resistance, and pulmonary artery diastolic pressure which appear within two hours post-infusion. These effects are similar to the physiologic changes seen in interleukin-2 (IL-2) therapy and septic shock (Marincola et al. (1993) J. Immunol. 13:282-288). These changes are sustained and augmented by subsequent IL-2 administration (Lee et al. (1989) J. Clin. Oncol. 7:7-20). IL-2 is a T-cell cytokine and its production is among the earliest events following stimulation of the T-cell receptor (TCR). The physiological changes observed in septic shock have been associated with elevated levels of TNF-α and IL-6, both of which are produced upon T-cell stimulation (Calandra et al. (1990) J. Infect. Dis. 161:982-987).

A comprehensive survey of the literature reveals that neither adoptive transfer of tumor-specific CTLs nor specific active immunotherapy with whole tumor cells or cell-derived preparations leads to eradication of tumors or long term survival in more than a minority of patients. It has been demonstrated in vitro that peptides have succeeded in priming T-cells where cell-derived preparations have failed (Cox et al. (1994) infra). Peptides that are expressed by the tumors of many individuals may be useful for immunotherapy, but the most generally applicable would be those that also are recognized by lymphocytes obtained from a large number of different cancer patients. Epitopes recognized by multiple CTL lines would be promising candidates for use in peptide-based anti-tumor vaccines.

In the absence of a reliable iterative method to identify tumor-specific antigens (TSAs), there is no way of assessing the limits of cross-reactivity. Identification of the antigens recognized by these tumor-specific CTLs is vital to the rational development of peptide-based anti-tumor vaccines. Moreover, for immunotherapy to be improved, epitopes recognized by tumor-specific CTLs must be identified. Thus, a two-pronged approach involving (1) identification and characterization of genes encoding tumor-specific antigens and (2) identification of the epitopes recognized by CTLs would facilitate enormously the development of immunotherapy-based cancer treatment strategies.

The foregoing discussion illustrates the need for improved methods for identifying tumor-specific antigens, as well as for identifying the epitopes recognized by CTLs. The present invention addresses this need and provides methods involving a combined approach for achieving these goals.

### SUMMARY OF THE INVENTION

The present invention provides a method for identifying epitopes and antigens recognized by immune effector cells and the polynucleotides that encode them. Thus, in one embodiment, the methods combine identifying the polynucleotides that encode sequence motifs of such antigens and identification of polynucleotides which are aberrantly expressed in the cells recognized by the immune effector cells. By comparison of these polynucleotide sequences, novel antigens that are recognized by immune effector cells can be identifed. This invention also provides a method for identifying and cloning the genes that encode the antigens as identified herein as well as methods of using genes and the proteins or polypeptides encoded by the genes.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Definitions

The practice of the present invention will employ, unless otherwise indicated, conventional techniques of molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)) and ANIMAL CELL CULTURE (R. I. Freshney, ed. (1987)).

As used herein, certain terms have the following defined meanings.

As used in the specification and claims, the singular form “a”, “an” and “the” include plural references unless the context clearly dictates otherwise. For example, the term “a cell” includes a plurality of cells, including mixtures thereof.

The term “antigen” is defined as a foreign substance that, upon introduction into a subject such as a verterbrate animal, stimulates the production of an immune response directed against the foreign substance. Thus, in one embodiment, an antigen is a polypeptide, protein or a fragment thereof containing an epitope, which induces an immune response in a subject.

The terms “antigen presenting cells” or “APCs” includes both intact, whole cells as well as other molecules which are capable of inducing the presentation and presentation of one or more antigens, preferably in association with class I MHC molecules. Examples of suitable APCs are discussed in detail below and include, but are not limited to, whole cells such as macrophages, dendritic cells, B cells purified MHC class I molecules complexed to β2-microglobulin and foster antigen presenting cells.

The terms “major histocompatibility complex” or “MHC” refers to a complex of genes (and the molecules encoded by them) that encode cell-surface molecules required for antigen presentation to T cells and for rapid graft rejection. In humans, the MHC complex is also known as the HLA complex. The proteins encoded by the MHC complex are known as “MHC molecules” and are classified into class I and class II MHC molecules. Class I MHC molecules include membrane heterodimeric proteins made up of an α chain encoded in the MHC associated noncovalently with β2-microglobulin. Class I MHC molecules are expressed by nearly all nucleated cells and have been shown to function in antigen presentation to CD8+ T cells. Class I molecules include HLA-A, -B, and -C in humans. Class II MHC molecules also include membrane heterodimeric proteins consisting of noncovalently associated α and β chains. Class II MHC are known to interact with CD4+ T cells and, in humans, include HLA-DP, -DQ, and DR. The term “MHC restriction” refers to a characteristic of T cells that permits them to recognize antigen only after it is processed and the resulting antigenic peptides are displayed in association with either a class I or class II MHC molecule.

The term “intact antigen presenting cell” is a living, viable cell with all functions intact, in contrast to presentation of antigen in a cell-free method, e.g., by binding an antigen to the major histocompatibility complex.

The term “foster antigen presenting cells” refers to any modified or naturally occurring cell (wild-type or mutant) with antigen presenting capability that is utilized in lieu of antigen presenting cells (“APC”) that normally contact the immune effector cells they are to react with. In other words, it is any functional APC that T cells would not normally encounter in vivo.

It has been shown that DCs provide all the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC”) class I or II protein on the surface of APCs. This interaction is necessary, but not sufficient, for T cell activation to occur. In fact, without the second type of signals, the first type of signals can result in T cell anergy. The second type of signals, called costimulatory signals, are neither antigen-specific nor MHC-restricted, and can lead to a full proliferation response of T cells and induction of T cell effector functions in the presence of the first type of signals.

Thus, the term “cytokine” refers to any of the numerous factors that exert a variety of effects on cells, for example, inducing growth or proliferation. Non-limiting examples of cytokines include, IL-2, stem cell factor (SCF), IL-3, IL-6, IL-12, G-CSF, GM-CSF, IL-1α, IL-11, MIP-1α, LIF, c-kit ligand, TPO, and flt3 ligand. Cytokines are commercially available from several vendors such as, for example, Genzyme Corp. (Framingham, Mass.), Genentech (South San Francisco, Calif.), Amgen (Thousand Oaks, Calif.) and Immunex (Seattle, Wash.). It is intended, although not always explicitly stated, that molecules having similar biological activity as wild-type or purified cytokines (e.g., recombinantly produced) are intended to be used within the spirit and scope of the invention and therefore are substitutes for wild-type or purified cytokines.

“Costimulatory molecules” are involved in the interaction between receptor-ligand pairs expressed on the surface of antigen presenting cells and T cells. One exemplary receptor-ligand pair is the B7 costimulatory molecules on the surface of DCs and its counter-receptor CD28 or CTLA4 on T cells (Freeman et al. (1993) Science 262:909-911; Young et al. (1992) J. Clin. Invest. 90: 229; and Nabavi et al. Nature 360:266). Other important costimulatory molecules are CD40, CD54, CD80, and CD86. These molecules are commercially available from vendors identified above.

The term “immune effector cells” refers to cells that specifically recognize an antigen present, for example on neoplastic or tumor cells. For the purposes of this invention, immune effector cells include, but are not limited to, B cells, monocytes, macrophages, NK cells and T cells such as cytotoxic T lymphocytes (CTLs), for example CTL lines, CTL clones, and CTLs from tumor, inflammatory, or other infiltrates. “T-lymphocytes” denotes lymphocytes that are phenotypically CD3+, typically detected using an anti-CD3 monoclonal antibody in combination with a suitable labeling technique. The T-lymphocytes of this invention are also generally positive for CD4, CD8, or both. The term “naïve” immune effector cells refers to immune effector cells that have not encountered antigen and is intended to be synonymous with unprimed and virgin. “Educated” refers to immune effector cells that have interacted with an antigen such that they differentiate into antigen-specific cells.

An “isolated” or “enriched” population of cells is “substantially free” of cells and materials with which it is associated in nature. By “substantially free” or “substantially purified” is meant at least 50% of the population are the desired cell type, preferably at least 70%, more preferably at least 80%, and even more preferably at least 90%.

The term “isolated” means separated from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eucaryotic cell in which it is produced in nature.

The terms “neoplastic cell”, “tumor cell”, or “cancer cell”, used either in the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by such procedures as CAT scan, magnetic resonance imaging (MRI), X-ray, ultrasound or palpation. Biochemical or immunologic findings alone may be insufficient to meet this definition.

The term “autogeneic”, or “autologous”, as used herein, indicates the origin of a cell. Thus, a cell being administered to an individual (the “recipient”) is autogeneic if the cell was derived from that individual (the “donor”) or a genetically identical individual. An autogeneic cell can also be a progeny of an autogeneic cell. The term also indicates that cells of different cell types are derived from the same donor or genetically identical donors. Thus, an effector cell and an antigen presenting cell are said to be autogeneic if they were derived from the same donor or from an individual genetically identical to the donor, or if they are progeny of cells derived from the same donor or from an individual genetically identical to the donor.

Similarly, the term “allogeneic”, as used herein, indicates the origin of a cell. Thus, a cell being administered to individual (the “recipient”) is allogeneic if the cell was derived from an individual not genetically identical to the recipient; in particular, the term relates to non-identity in expressed MHC molecules. An allogeneic cell can also be a progeny of an allogeneic cell. The term also indicates that cells of different cell types are derived from genetically non-identical donors, or if they are progeny of cells derived from genetically non-identical donors. For example, an APC is said to be allogeneic to an effector cell if they are derived from genetically non-identical donors.

The term “sequence motif” refers to a pattern present in a group of molecules. For instance, in one embodiment, the present invention provides for identification of a sequence motif of amino acids among peptides that comprise an epitope. In this embodiment, a typical pattern may be identified by characteristic amino acid residues, such as hydrophobic, hydrophilic, basic, acidic, and the like.

The term “peptide” is used in its broadest sense to refer to a compound of two or more subunit amino acids, amino acid analogs, or peptidomimetics. The subunits may be linked by peptide bonds. In another embodiment, the subunit may be linked by other the bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

As used herein, “solid phase support” is not limited to a specific type of support. Rather a large number of supports are available and are known to one of ordinary skill in the art. Solid phase supports include silica gels, resins, derivatized plastic films, glass beads, cotton, plastic beads, alumina gels. A suitable solid phase support may be selected on the basis of desired end use and suitability for various synthetic protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories); polystyrene resin grafted with polyethylene glycol (TentaGel®, Rapp Polymere, Tubingen, Germany) or polydimethylacrylamide resin (obtained from Milligen/Biosearch, California). In a preferred embodiment for peptide synthesis, solid phase support refers to polydimethylacrylamide resin.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. For example, “polynucleotide” includes single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

The term “aberrantly expressed” refers to polynucleotide sequences in a cell or tissue that are differentially expressed (either over-expressed or under-expressed) when compared to a different cell or tissue whether or not of the same tissue type, i.e., lung tissue versus lung cancer tissue.

A “subject” is a vertebrate, preferably a mammal, more preferably a human. Mammals include, but are not limited to, murines, simians, humans, farm animals, sport animals, and pets.

An “effective amount” is an amount sufficient to effect beneficial or desired results. An effective amount can be administered in one or more administrations, applications or dosages. Methods of administering are well known in the art and include, but are not limited to injection, aerosol administration and intraperateneal.

“Oligonucleotide” refers to polynucleotides of between about 5 and about 100 nucleotides of single- or double-stranded DNA. Oligonucleotides are also known as oligomers or oligos and may be isolated from genes, or chemically synthesized by methods known in the art.

A “primer” refers to an oligonucleotide, usually single-stranded, that provides a 3′-hydroxyl end for the initiation of enzyme-mediated nucleic acid synthesis. The primer sequence need not reflect the exact sequence of the template.

“PCR primers” refer to primers used in “polymerase chain reaction” or “PCR,” a method for amplifying a DNA base sequence using a heat-stable polymerase such as Taq polymerase, and two oligonucleotide primers, one complementary to the (+)-strand at one end of the sequence to be amplified and the other complementary to the (−)-strand at the other end. Because the newly synthesized DNA strands can subsequently serve as additional templates for the same primer sequences, successive rounds of primer annealing, strand elongation, and dissociation produce exponential and highly specific amplification of the desired sequence. (See, e.g., PCR 2: A PRACTICAL APPROACH, supra). PCR also can be used to detect the existence of the defined sequence in a DNA sample.

A “sequence tag” or “SAGE tag” is a short sequence, generally under about 20 nucleotides, that occurs in a certain position in messenger RNA. The tag can be used to identify the corresponding transcript and gene from which it was transcribed. A “ditag” is a dimer of two sequence tags.

The term “cDNAs” refers to complementary DNA, that is mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules which can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

As used herein, the terms “restriction endonucleases” and “restriction enzymes” refer to bacterial enzymes which bind to a specific double-stranded DNA sequence termed a recognition site or recognition nucleotide sequence, and cut double-stranded DNA at or near the specific recognition site. “Type IIS” restriction endonucleases are those which cleave at a defined distance (up to 20 bases away) from their recognition sites. Endonucleases will be known to those of skill in the art (see for example, CURRENT PROTOCOLS IN MOLECULAR BIOLOGY, Vol. 2, ed. (Ausubel et al. (1995)), GREENE PUBLISH. Assoc. & WILEY INTERSCIENCE, Unit 3.1.15; New England Biolabs Catalog (1995)).

The term “combining in a combinatorial fashion” means combining the elements in every possible combination.

A “composition” is intended to mean a combination of active agent and another compound or composition, inert (for example, a detectable agent, carrier, solid support or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

“Host cell” or “recipient cell” is intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation. The cells may be procaryotic or eucaryotic, and include but are not limited to bacterial cells, yeast cells, animal cells, and mammalian cells, e.g., murine, rat, simian or human.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

This invention provides various methods of identifying one or more polynucleotide fragments of one or more genes encoding antigens recognized by immune effector cells. The method comprises:
(a) providing a first cell that expresses an antigen recognized by the immune effector cell and having an identified major histocompatibility complex (MHC) restriction and one or more second cells having a compatible major histocompatibility complex (MHC) to the first cell but which does not express antigen;
(b) Identifying polynucleotides encloding a peptide, a sequence motif in the antigen displayed by antigen presenting cells and recognized by the immune effector cell;
(c) identifying polynucleotides which are aberrantly expressed by the first cells as compared one or more to second cells; and
(d) comparing the polynucleotides identified in step (c) with the polynucleotides encoding the peptide sequence motifs identified in step (b) to identify the fragment of the gene encoding the antigen recognized by the immune effector cell. The method of step, (c) (above) may, in one embodiment, be performed prior to step (b). The first and second cells are animal cells that include, but are not limited to human, murine, rat or simian cells. They can be antologons or alogeneic as defined above.

This method identifies polynucleotides that have the potential to encode the peptide sequences or motifs that are antigenic or a fragment of the antigenic protein or polypeptide. Thus, the method further encompasses confirmation that the expression product encodes the antigen of interest by introducing into a cell the polynucleotide under conditions that it is expressed and presented by an APC by a compatible MHC. Methods for recognition by immune effector cells are provided below.

The “first cell” must satisfy two criteria: 1) it must express an antigen recognized by an immune effector cell; and 2) it must have an identified major histocompatibility complex restriction. The first and second cell populations are pre-selected to have compatible MHC restriction. Methods of identifying and comparing MHC are well known in the art and are described in Allen, M. et al. (1994) Human Imm. 40:25-32; Santamaria P. et al. (1993) Human Imm. 37:39-50 and Hurley C. K. et al. (1997) Tissue Antigens 50:401-415. Methods of determining whether the antigen is recognized by an immune effector cell are well known in the art, and include methods such as ³H-thymidine incorporation; metabolic activity detected by conversion of MTT to formazan blue; increased cytokine mRNA expression; increased cytokine protein production; and chromium release by target cells.

Any cell or population of cells that presents antigen recognized by immune effector cells is useful and within the scope of this invention. Such cells include, but are not limited to antigen presenting cells (defined above), cells having a purified MHC class I molecule complexed to a β2-micro globulin, dendritic cells, intact antigen presenting cells or foster antigen presenting cells. Methods for isolating and culturing these cells are provided in the Experimental Section, infra.

Immune effector cells (defined above) recognize the APCs. Immune effector cells for the purpose of this invention include, but are not limited to CTLs, and cells isolated from a site of viral infection, a site of autoimmune infiltration, a site of transplantation rejection, a site of inflammation, a site of lymphocyte infilitration and a site of leukocyte infiltration. Suitable CTLs include, but are not limited to polyclonal T cells isolated from one individual, polyclonal T cells isolated from two or more individuals sharing the same MHC restriction, two or more CTLs or any combination thereof. Methods for isolating and culturing these cells are provided in the Experimental Section, infra.

The second cell that does not express antigen can be in one embodiment, a foster antigen presenting cell that lacks antigen processing activity and expresses MHC molecules free of bound peptides.

After preselection of the first and second cell(s), the polynucleotides that encode a peptide sequence motif in the antigen displayed by the antigen presenting cells (the first cell population) is then identified. In one embodiment, the peptide sequence motif is first identified, from which the polynucleotide is then derived. Any of the various methods that identify peptide sequence motifs in antigens recognized by immune effector cells are useful to perform this step of the invention. These methods are described infra in “Identification of the Epitopes Recognized by CTLs”. Briefly, such methods include, but are not limited to the “phage method” (Scott and Smith (1990) Science 249:386-390; Cwirla et at. (1990) PNAS 87:6378-6382; and Devlin et al. (1990) Science 249:404-406), the Geysen method (Geysen et al. (1986) Molecular Immunology 23:709-715; and Geysen et al. (1987) J. Immunologic Method 102:259-274), the method of Fodor et al. (11991) Science. 251:767-773), methods to test peptides that are agonists or antagonists as described in Furka et al. (1988) 14th International Congress of Biochemistry, Volume 5. Abstract FR:013; Furka, (1991) Int. J. Peptide Protein Res. 37:487-493); Houghton (U.S. Pat. No. 4,631,211 issued December 1986); and Rutter et al. (U.S. Pat. No. 5,101,175, issued Apr. 23, 1991), the method utilizing synthetic libraries (Needels et al. (1993) PNAS 90:107004; Ohlmeyer et al. (1993) PNAS 90:10922-10926; and Lam et al., International Patent Publication No. WO 92/00252), the method that utilizes indexed combinatorial peptide displays (Ohlmeyer et al. (1993) PNAS 90:10922-26) and the pepscan technique by Van der Zee (1989) Eur. J. Immunol. 19:4347. In one embodiment, the method utilizes SPHERE (described in PCT WO97/35035 and in detail infra in the Experimental Section entitled “Identification of Sequence Motifs in Antigens Recognized by Immune Effector Cells”). Briefly, SPHRE is an empirical screening method for the identification of MHC Class I-restricted CTL epitopes that utilizes peptide libraries synthesized on a solid support (e.g., plastic beads) where each bead contains approximately 200 picomoles of a unique peptide that can be released in a controlled manner. The synthetic peptide library is tailored to a particular HLA restriction by fixing anchor residues that confer high-affinity binding to a particular HLA allele (e.g., HLA-A2) but contain a variable TCR epitope repertoire by randomizing the remaining positions. Roughly speaking, 50 96-well plates with 10,000 beads per well will accommodate a library with a complexity of approximately 5×10⁷. In order to minimize both the number of CTL cells required per screen and the amount of manual manipulations, the eluted peptides can be further pooled to yeild wells with any desired complexity. Based on experiments with soluble libraries, it should be possible to screen 10⁷ peptides in 96-well plates (10,000 peptides per well) with as few as 2×10⁶ CTL cells. After cleaving a percentage of the peptides from the beads and incubating them with ⁵¹Cr-labeled APCs (e.g., foster antigen presenting cells or T2 cells) and the CTL line(s), peptide pools containing reactive species can be determined by measuring ⁵¹Cr-release according to standard methods known in the art. Alternatively, cytokine production (e.g., interferon-γ) or proliferation (e.g., incorporation of ³H-thymidine) assays may be used. After identifying reactive 10,000-peptide mixtures, the beads corresponding to those mixtures are separated into smaller pools and distributed to new 96-well plates (e.g., 100 beads per well). An additional percentage of peptide is released from each pool and reassayed for activity by one of the methods listed above. Upon identification of reactive 100-peptide pools, the beads corresponding those peptide mixtures are redistributed at 1 bead per well of a new 96-well plate. Once again, an additional percentage of peptide is released and assayed for reactivity in order to isolate the single beads containing the reactive library peptides. The sequence of the peptides on individual beads can be determined by sequencing residual peptide bound to the beads by, for example, N-terminal Edman degradation or other analytical techniques known to those of skill in the art.

Degenerate polynucleotide sequences that encode the peptide motif or motifs are then determined.

Prior to or subsequent to identifying polynucleotides that encode sequence motifs, polynucleotides that are aberrantely or differentially expressed between the first and second cells are identified. Many methods are known in the art to identify differentially expressed polynucleotides and each can be used to provide these polynucleotides. As used herein, the term “polynucleotide fragment” includes SAGE tags (defined above) as well as any other nucleic acid obtained from any methods that yeild quantitative/comparative gene expression data. These methods are described in detail infra in “Methods for Identification and Characterization of Genes Encoding Tumor-Specific Antigens”. Such methods include, but are not limited to cDNA subtraction, differential display and expressed sequence tag methods. Techniques based on cDNA subtraction or differential display can be quite useful for comparing gene expression differences between two cell types (Hedrick et al. (1984) Nature 308:149 and Lian and Pardee (1992) Science 257:967). The expressed sequence tag (EST) approach is another valuable tool for gene discovery (Adams et al. (1991) Science 252:1651), like Northern blotting, RNase protection, and reverse transcriptase-polymerase chain reaction (RT-PCR) analysis (Alwine et al. (1977) PNAS 74:5350; Zinn et al. (1983) Cell 34:865; and Veres et al. (1987) Science 237:415). A futher method is differential display coupled with real time PCT and representational difference analysis (Lisitisyn and Wigler (1995) Meth. Enzymol. 254:291-304). Another approach is the technology known as Serial Analysis of Gene Expression (SAGE, described in U.S. Pat. No. 5,695,937). Using SAGE, one can identify and provide complementary deoxyribonucleic acid (cDNA) polynucleotides from an antigen expressing cell recognized by the immune effector cells. Using SAGE again, one can identify and provide cDNA polynucleotides from cells having a compatible major histocompatability complex (MHC) to the cells of step (a) but which do not express antigen. The information obtained from these two sets are then analyzed to identify the cDNAs that are aberrantly expressed by the first cells as compared to the second cells.

The polynucleotides identified in steps (b) and (c) are compared to identify those polynucleotides or the polynucleotides corresponding to the genes, or fragments of the genes, that are common to the polynucleotides of the first and second cells. The common polynucleotides represent fragments of the genes that encode antigens recognized by the immune effector cells. The biological activity of the peptides encoded by the invention polynucleotides can be confirmed using methods described herein.

In one embodiment of this invention, step (b) of the method utilizes SPHERE, which in one embodiment is combined with SAGE for step (c). SAGE defines a portion of a gene very close to the polyA signal. Accordinaly, the polynucleotide identified by the SAGE method (step (c), above) is usually very close to the 3′ polyA tail.

In contrast, the SPHERE process defines an epitope which is most likely to be located 5′ of the sequence identified by SAGE or in some cases, other methods, because it is highly unlikely to have a coding sequence at the 3′ end near the polyA tail. Therefore, to identify and compare the polynucleotides of steps (b) and (c), a primer is designed from the SAGE tag to run in a 3′ to 5′ direction relative to the sense strand. Degenerate primers corresponding to the SPHERE epitope (which is a peptide sequence) are designed that take into consideration all possible nucleotide combinations that will encode the peptide. These primers run in 5′ to 3′ relative to the sense strand of the antigen polynucleotide.

A library of 5′ PCR primers also can be generated that correspond to the polynucleotide sequences identified to be the peptide motifs which the specified immune effector cell, e.g., a CTL, recognize and bind. A library of expressed sequence tags (ESTs) can be generated from the cells expressing the antigen. By screening for those tags that are differentially expressed in the antigen expressing samples, a handful of cDNAs can be identified which correspond to candidate antigens. 3′ primers are then generated based on these sequences.

The 5′ PCR primers are mixed in a combinatorial fashion with the 3′PCR primers such that all possible combinations that can be used in PCR reactions occur. The template for the amplification reactions is total cDNA from the sample cell, e.g., a tumor cell sample which expresses the antigen motif recognized by the immune effector cell. By examining the patterns of the PCR products generated, it is possible to discern, for example: a) which SAGE sequences or tags correspond to cDNAs encoding tumor antigens; b) how many of the epitopes identified by SPHERE lie within any one antigen as opposed to different antigens; c) the relative positions of epitopes with respect to one another should a tumor antigen contain more that one epitope that has been identified via SPHERE; d) which SAGE tags can be dispensed with based on their failure to promote the generation of a PCR product provided all the 5′ PCR primers are accounted for.

As noted above, these embodiments require that the sequences of the SAGE or sequence tags be used to generate 3′ PCR primers. Anchored PCR can be conducted using a cDNA library developed from the antigen-containing or presenting cell recognized by the immune effector cell. The cDNA library can be one in which universal linkers are appended onto the 3′ and 5′ ends of each cDNA (in a manner similar to the commercially available Marathon cDNA libraries). Thus, it is possible to use a 5′ PCR primer corrsponding to the 5′ universal linker and a 3′ PCR primer based on the SAGE tag sequence to PCR amplify specific cDNA segments corresponding to potential antigens. The oligonucleotides that are based on the amino acid sequences of the epitopes identified by SPHERE are used to either 1) hybridize to the PCR products generated above that correspond to partial cDNAs or 2) PCR amplify again.

It is possible to further modify and automate this method by rapidly linking messages (mRNA) expressed in tumor cells (identified by SAGE analysis) with T cell epitopes recognized by CTLs (identified by SPHERE) to identify candidate tumor antigens without having to go to the trouble of converting each.

SAGE tag to its corresponding cDNA or having to sequence every candidate cDNA to ascertain whether they encode a potential tumor antigen.

LINKAGE OF SAGE AND SPHERE

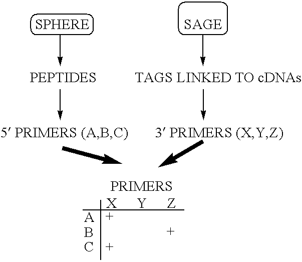

The above method is useful to identify an antigen expressed on any cell that is recognized by an immune effector cell, e.g., a neoplastic or tumor cell. Alternatively, the antigen recognized by the immune effector cell is expressed at a site of viral infection, a site of autoimmune infiltration, a site of transplantation rejection, a site of inflammation or a site of lymphocyte or leukocyte infiltration. The antigen recognized by the immune effector cell at the site of viral infection are isolated from peripheral blood lymphoctyes. Alternatively, isolate from an arthritic joint is used as the cell samples to identify genes encoding antigens recognized by immune effector cells at the site of autoimmune infiltration. Tissue from the site of transplant rejection can be used for cell samples to identify genes encoding antigens at the site of transplantation rejection. These samples can be isolated from “subjects” as defined above.

The partial cDNA clone (i.e., the PCR product) generated above can be used in one embodiment to clone out a full length cDNA encoding an antigen using methods well-known in the art, for example expression cloning as set forth in Kawakami, Y. et al. (1994) PNAS 91 (9):3515-3519. In this approach, mRNA is isolated from the antigen expressing cells, and the mRNA is converted into cDNA. The resulting DNA fragments are inserted into plasmids or other appropriate expression vectors. After an amplification step in bacteria or another host cell, the plasmid DNA is transfected into and appropriate host cell, e.g. COS cells (a permanent cell culture derived from African green monkey kidney cells) together with plasmid DNA encoding the appropriate HLA molecule. The antigen-specific immune effector clone is then added to the transfected COS cells. If some of the COS cells express the antigen (because they received the right cDNA), the immune effector cells will be stimulated to produce a cytokine, such as, for example IFNγ or tumor necrosis factor (TNF), which can be detected in the culture medium. In order to screen all the mRNA molecules present in the tumor, approximately 10⁵ plasmids have to be tested, in pools of 100 different molecules. The pool of plasmid DNA found to be positive for T-cell stimulation can then be divided and the transfection procedure repeated until the preparation of a single species of plasmid DNA is found that can transfer the expression of the antigen.

The isolated polynucleotides and the genes corresponding to the isolated polynucleotides are also provided by this invention. As used herein, the term “polynucleotide” encompasses DNA, RNA and nucleic acid mimetics. In addition to the polynucleotides and their complements, this invention also provides the anti-sense polynucleotide stand, e.g. antisense RNA to these sequences or their complements. One can obtain an antisense RNA using the sequences provided by this invention and the methodology described in Vander Krol et al. (1988) BioTechniques 6:958. Further provided by this invention are polynucleotides that are substantially identical (at least 50%, more preferably at least 75%, and most preferably at least 90% or greater than 95% homologous as determined using sequence homology programs or hybridization techniques) to the isolated polynucleotides and genes provided by this invention.

In a further embodiment, the polynucleotide or gene sequence can also be compared to a sequence database, for example, using a computer method to match a sample sequence with known sequences. Sequence identity can be determined by a sequence comparison using, i.e., sequence alignment programs that are known in the art, such as those described in CURRENT PROTOCOLS IN MOLECULAR BIOLOGY (F. M. Ausubel et al., eds., 1987) Supplement 30, section 7.7.18, Table 7.7.1. A preferred alignment program is ALIGN Plus (Scientific and Educational Software, Pennsylvania), preferably using default parameters, which are as follows: mismatch =2; open gap=0; and extend gap=2. Another preferred program is the BLAST program for alignment of two nucleotide sequences, using default parameters as follows: open gap=50; extension gap−2 penalties; gap×dropoff=0; expect 10; word size=11. The BLAST program is available at the following Internet address: http://www.ncbi.nlm.nih.gov. As noted above, alternatively, hybridization under conditions of high, moderate and low stringency can also indicate degree of sequence identity.

The polynucleotides can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. One of skill in the art can employ a fluorescent label or an enzyme tag, such as urease, alkaline phosphatase or peroxidase, instead of radioactive or other environmental undesirable reagents. In the case of enzyme tags, calorimetric indicator substrates are known which can be employed to provide a means visible to the human eye or spectrophotometrically, to identify specific hybridization with complementary nucleic acid-containing samples. Briefly, this invention further provides a method for detecting a single-stranded or its complement, by contacting target single-stranded polynucleotides with a labeled, single-stranded polynucleotide (a probe) which is at least 4, and more preferably at least 5 or 6 and most preferably at least 10 contingent nucleotides of this invention under conditions permitting hybridization (preferably moderately stringent hybridization conditions) of complementary single-stranded polynucleotides, or more preferably, under highly stringent hybridization conditions. Hybridized polynucleotide pairs are separated from un-hybridized, single-stranded polynucleotides. The hybridized polynucleotide pairs are detected using methods well known to those of skill in the art and set forth, for example, in Sambrook et al. (1989) supra. The polynucleotides can be provided in kits with appropriate reagents and instructions for their use as probes or primers.

The polynucleotides of this invention can be replicated using PCR. PCR technology is the subject matter of U.S. Pat. Nos. 4,683,195, 4,800,159, 4,754,065, and 4,683,202 and described in PCR: THE POLYMERASE CHAIN REACTION (Mullis et al. eds, Birkhauser Press, Boston (1994)) and references cited therein.

Alternatively, one of skill in the art can use the sequences provided herein and a commercial DNA synthesizer to replicate the DNA. Accordingly, this invention also provides a process for obtaining the polynucleotides of this invention by providing the linear sequence of the polynucleotide, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the polynucleotide into a suitable replication vector and insert the vector into a suitable host cell (procarybtic or eucaryotic) for replication and amplification. The DNA so amplified can be isolated from the cell by methods well known to those of skill in the art. A process for obtaining polynucleotides by this method is further provided herein as well as the polynucleotides so obtained.

RNA can be obtained by first inserting a DNA polynucleotide into a suitable host cell. The DNA can be inserted by any appropriate method, e.g., by the use of an appropriate gene delivery vehicle (e.g., liposome, plasmid or vector) or by electroporation. When the cell replicates and the DNA is transcribed into RNA; the RNA can then be isolated using methods well known to those of skill in the art, for example, as set forth in Sambrook et al. (1989) supra. For instance, mRNA can be isolated using various lytic enzymes or chemical solutions according to the procedures set forth in Sambrook et al. (1989) supra or extracted by nucleic acid-binding resins following the accompanying instructions provided by manufactures.

The polynucleotides can be used as probes or primers. Host cells containing polynucleotides of this invention also are within the scop of this invention. It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. Preferably, a probe useful for detecting the aforementioned mRNA is at least about 80% identical to the homologous region of comparable size contained in the polynucleotides of this invention. More preferably, the probe is 85% identical to the corresponding gene sequence after alignment of the homologous region; even more preferably, it exhibits 90% identity.

These probes can be used in radioassays (e.g. Southern and Northern blot analysis) to detect or monitor various cells or tissue containing these cells. The probes also can be attached to a solid support or an array such as a chip for use in high throughput screening assays for the detection of expression of the gene corresponding to one or more polynucleotide(s) of this invention. Accordingly, this invention also provides at least one probe as defined above of the transcripts or the complement of one, of these sequences, attached to a solid support such as a chip for use in high throughput screens.

The polynucleotides of this invention also can be used for comparison to known and unknowns sequences using a computer-based method to match a sample sequence with known sequences. Thus, this invention also provides the polynucleotides of this invention in a computer database or, in computer readable form, including applications utilizing the internet.

A linear search through such a database may be used. Alternatively, the polynucleotide sequence can be converted into a unique numeric representation. The comparison aspects may be implemented in hardware or software, or a combination of both. Preferably, these aspects of the invention are implemented in computer programs executing on a programable computer comprising a processor, a data storage system (including volatile and non-volatile memory and/or storage elements), at least one input device, and at least one output device. Data input through one or more input devices for temporary or permanent storage in the data storage system includes sequences, and may include previously generated polynucleotides and codes for known and/or unknown sequences. Program code is applied to the input data to perform the functions described above and generate output information. The output information is applied to one or more output devices, in known fashion.

Each such computer program is preferably stored on a storage media or device (e.g., ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

The polynucleotides of the present invention also can serve as primers for the detection of genes or gene transcripts that are expressed in APC, for example, to confirm transduction of the polynucleotides into host cells. In this context, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase. A preferred length of the primer is the same as that identified for probes, above.

The invention further provides the isolated polynucleotide operatively linked to a promoter of RNA transcription, as well as other regulatory sequences for replication and/or transient or stable expression of the DNA or RNA. As used herein, the term “operatively linked” means positioned in such a manner that the promoter will direct transcription of RNA off the DNA molecule. Examples of such promoters are SP6, T4 and T7. In certain embodiments, cell-specific promoters are used for cell-specific expression of the inserted polynucleotide. Vectors which contain a promoter or a promoter/enhancer, with termination codons and selectable marker sequences, as well as a cloning site into which an inserted piece of DNA can be operatively linked to that promoter are well known in the art and commercially available. For general methodology and cloning strategies, see GENE EXPRESSION TECHNOLOGY (Goeddel ed., Academic Press, Inc. (1991)) and references cited therein and VECTORS: ESSENTIAL DATA SERIES (Gacesa and Ramji, eds., John Wiley & Sons, N.Y. (1994)), which contains maps, functional properties, commercial suppliers and a reference to GenEMBL accession numbers for various suitable vectors. Preferable, these vectors are capable of transcribing RNA in vitro or in vivo.

Expression vectors containing these nucleic acids are useful to obtain host vector systems to produce proteins and polypeptides. It is implied that these expression vectors must be replicable in the host organisms either as episomes or as an integral part of the chromosomal DNA. Suitable expression vectors include plasmids, viral vectors, including adenoviruses, adeno-associated viruses, retroviruses, cosmids, etc. Adenoviral vectors are particularly useful for introducing genes into tissues in vivo because of their high levels of expression and efficient transformation of cells both in vitro and in vivo. When a nucleic acid is inserted into a suitable host cell, e.g., a procaryotic or a eucaryotic cell and the host cell replicates, the protein can be recombinantly produced. Suitable host cells will depend on the vector and can include mammalian cells, animal cells, human cells, simian cells, insect cells, yeast cells, and bacterial cells constructed using well known methods. See Sambrook et al. (1989) supra. In addition to the use of viral vector for insertion of exogenous nucleic acid into cells, the nucleic acid can be inserted into the host cell by methods well known in the art such as transformation for bacterial cells; transfection using calcium phosphate precipitation for mammalian cells; or DEAE-dextran; electroporation; or microinjection. See Sambrook et al. (1989) supra for this methodology. Thus, this invention also provides a host cell, e.g. a mammalian cell, an animal cell (rat or mouse), a human cell, or a procaryotic cell such as a bacterial cell, containing a polynucleotide encoding a protein or polypeptide or antibody.

When the vectors are used for gene therapy in vivo or ex vivo, a pharmaceutically acceptable vector is preferred, such as a replication-incompetent retroviral or adenoviral vector. Pharmaceutically acceptable vectors containing the nucleic acids of this invention can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6 (Miller, A. D. et al. (1989) BioTechniques 7:980-990). The methodology of using replication-incompetent retroviruses for retroviral-mediated gene transfer of gene markers is well established (Correll et al. (1989) PNAS 86:8912; Bordignon (1989) PNAS 86:8912-52; Culver K. (1991) PNAS 88:3155; and Rill D. R. (1991) Blood 79 (10):2694-700.

The polynucleotides, genes and encoded peptides and proteins can be further cloned and expressed in vitro or in vivo. Expression and cloning vectors as well as host cells containing these polynucleotides and genes are claimed herein as well as methods of administering them to a subject in an effective amount. Peptides corresponding to these sequences can be generated by recombinant technology and they may be administered to a subject as a vaccine or alternatively, introduced into APC which in turn, are administered in an effective amount to a subject. The genes may be used to produce proteins which in turn may be used to pulse APC. The APC may in turn be used to expand immune effector cells such as CTLs. The pulsed APC and expanded effector cells can be used for immunotherapy by administering an effective amount of the composition to a subject.

In another preferred embodiment, the methods of this invention are used to monitor expression of the genes which specifically hybridize to the probes of this invention in response to defined stimuli, such as a drug.

In one embodiment, the hybridized nucleic acids are detected by detecting one or more labels attached to the sample nucleic acids. The labels may be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g. fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

The polynucleotide also can be modified prior to hybridization to a high density probe array in order to reduce sample complexity thereby decreasing background signal and improving sensitivity of the measurement using the methods disclosed in WO 97/10365. They also can be attached to a chip for use in diagnostic and analytical assays. Results from the chip assay are typically analyzed using a computer software program. See, for example, EP 0717 113 A2 and WO 95/20681. The hybridization data is read into the program, which calculates the expression level of the targeted gene(s). This figure is compared against existing data sets of gene expression levels for diseased and healthy individuals.

Also provided by this invention are antibodies that specifically react with the peptides and proteins of this invention. Such antibodies include, but are not limited to polyclonal antibodies, monoclonal antibodies, chimeric antibodies, humanized antibodies and antibody fragments. These can be combined with detectable labels and used to identify antigens and fragments thereof using well known methods. Alternatively, they can be combined with pharmaceutically acceptable carriers and administered therapeutically to a subject in need of such treatment. Kits containing the antibodies, reagents and instructions for use are further provided by this invention.

Thus, it should be understood, although not always explicitely stated, that the compositions of this invention can be combined with a pharmaceutically acceptable carrier prior to administration or combined with a carrier for in vitro use. These in vitro carriers, include, but are not limited, beads for use in cell separation methodologies.

The following examples are intended to illustrate, but not limit, the invention as described herein.

Methods for Identification and Characterization of Genes Encoding Tumor-Specific Antigens

Most tumor antigens that have been studied are proteins, especially glycoproteins. The increased understanding of the nature of presentation of peptides by major histocompatibility (MHC) molecules to cytotoxic T lymphocytes (CTLs) indicates that most, if not all, antigens that stimulate CTLs are proteins. These need not be surface or structural proteins of the cell, because peptides from cytoplasmic proteins can be processed in the endoplasmic reticulum, where the MHC molecules are assembled, and become associated with the MHC molecules there. The MHC molecules are then transported to the surface of the cell with the tumor-associated peptides already bound. Nevertheless, gangliosides and saccharides can elicit humoral responses, and can protect mice against tumors bearing those sugars. CTLs may recognize a portion of the saccharide structure surrounding the core peptide, as are present on mucins of adenocarcinomas.

While tumor-specific antigenic determinants' have not been extensively elucidated for many types of cancers, the sequence of a peptide called MAGE-1, which is a target of human CTLs directed against melanomas, has been described. Van der Bruggen et al. (1991) Science 254:1643; Other target epitopes have been identified, for example, the melanoma-specific antigen MAGE-3, the adenocarcinoma-associated antigen MUC-1, and the HER-2/neu extracellular domain protein associated with breast and ovarian cancers.

The reasons why tumor cells may express tumor-specific antigens are beginning to be understood. For example, TSAs may be the result of the processes of carcinogenesis, which are generally thought to stem from damage to a large number of genes, some of which have a role in the molecular mechanisms regulating cell growth and division. This damage results in uncontrolled cellular proliferation that defines the transformed cell. Thus, possible origins of TSAs include self proteins (such as fetal antigens) oncogene products (including fusion proteins), mutated tumor suppressor gene products, other mutated cellular proteins, or foreign proteins such as viral gene products. Nonmutated cellular proteins may also be antigenic if they are expressed aberrantly (e.g., in an inappropriate subcellular compartment) or in supernormal quantities. Given the numerous steps of cellular transformation and sometimes bizarre genotypes observed in cancer cells, it could be argued that tumor cells are likely to contain many new antigens potentially recognizable by the immune system.

Reports of shared tumor antigens are frequent in the literature. In the case of melanoma, there is recent evidence that the same T-cell-defined tumor antigens are expressed by independent human melanoma suggesting that transformation-associated events may give rise to recurrent expression of the same tumor antigen in different tumors of related tissue and cellular origin (Sahasrabudhe et al. (1993) J. Immunol. 151:6302-6310; Shamamian et al. (1994) Cancer Immunol. Immunother. 39:73-83; Cox et al. (1994) Science 264:716; Peoples et al. (11993) J. Immunol. 151:5481-5491; Jerome et al. (1991) Cancer Res. 51:2908-2916; and Morioke et al. (1994) J. Immunol. 153:5650-5658). Previous studies in animal models have, in contrast, suggested that most chemical and ultraviolet radiation-induced tumors are antigenically diverse and that tumor rejection antigen may be generated by random mutation (Srivastava et al. (1986) PNAS 83:3407-3411). However, it is highly improbable that a completely random process would give rise to shared antigens even in very closely related tumors. This data supports the possibility that specific anti-tumor immunotherapies, such as vaccines, may be active against more than one form of cancer and that the same vaccine may be effective against independently derived tumors of the same type.

Identification of TSAs can be accomplished by a variety of methods, including cDNA subtraction, differential display and expressed sequence tag methods. Techniques based on cDNA subtraction or differential display can be quite useful for comparing gene expression differences between two cell types (Hedrick et al. (1984) Nature 308:149; and Lian and Pardee (1992) Science 257:967), but provide only a partial picture, with no direct information about abundance. The expressed sequence tag (EST) approach is a valuable tool for gene discovery (Adams et al. (1991) Science 252:1651), but like Northern blotting, RNase protection, and reverse transcriptase-polymerase chain reaction (RT-PCR) analysis (Alwine et al. (1977) PNAS 74:5350; Zinn et al. (1983) Cell 34:865; and Veres et al. (1987) Science 237:415), only evaluates a limited number of genes at a time. Another approach is the use of SAGE, a technique that allows a rapid, detailed analysis of thousands of transcripts.

SAGE is based on two principles. First, a short nucleotide sequence tag (9 to 10 bp) contains sufficient information content to uniquely identify a transcript provided it is isolated from a defined position within the transcript. For example, a sequence as short as 9 bp can distinguish 262,144 transcripts (Fields et al. (1994) Nature Genet. 7:345) given a random nucleotide distribution at the tag site, whereas current estimates suggest that even the human genome only encodes about 80,000 transcripts (Fields et al. supra). Second, concatenation of short sequence tags allows the efficient analysis of transcripts in a serial manner by sequencing of multiple tags within a single clone. As with serial communication by computers, wherein information is transmitted as a continuous string of data, serial analysis of the sequence tags requires a means to establish the register and boundaries of each tag.

In addition, to providing quantitative information on the abundance of known transcripts, SAGE can be used to identify novel expressed genes. SAGE can provide both quantitative and qualitative data about gene expression. The combination of different anchoring enzymes with various recognition sites and type IIS enzymes with cleavage sites 5 to 20 bp from their recognition elements lends great flexibility to this strategy. In principle, SAGE can allow a direct readout of expression in any given cell type or tissue. SAGE is also useful for the comparison of gene expression patterns in various developmental and disease states. Adoption of this technique to an automated sequencer would allow the analysis of over 1,000 transcripts in a single 3 hour run (Deininger et. al. (0.1981) J. Mol. Biol. 151:17).

However, SAGE alone does not readily distinguish between “housekeeping” genes and those genes expressed due to a particular disease state.

Identification of the Epitopes Recognized by CTLs

Epitopes recognized by CTLs are 8-10 amino acid peptides derived from cellular proteins that are endocytically processed and presented on the tumor cell surface by major histocompatability complex (MHC) class I and class II glycoproteins. MHC class I molecules are expressed in virtually all nucleated cells and the combination of peptide and MHC molecule is specifically recognized by the appropriate T-cell receptors (TCRs). T-cells in the presence of antigen presenting cells and their corresponding antigen proliferate and acquire potent cytolytic activity.

There are several obstacles which contribute to the difficulty of analyzing MHC-associated peptides by classical means. Current protocols involve isolating and assaying extremely pure MHC molecules from antigen presenting cells. Prior to peptide extraction, all contaminating proteinaceous material must be removed (this includes low molecular weight contaminants that normally escape detection by routine methods used to analyze protein purity such as SDS-PAGE) (Chicz and Urban (1994) Immunol. Today 1-5:155-160).

Briefly, immunoaffinity purification yields approximately 0.5-1 mg of HLA molecules per gram (11 culture) of B-cell lymphocytes (yields from B-cells are significantly higher than those obtained from primary explant tissues). Since the bound peptide is only 8-10 amino acids long, 1 mg of MHC contains 16 pmol of extractable peptide. Furthermore, the efficiency of peptide extraction is typically 75-80%. Thus, 1 mg of MHC usually yields 13 pmol of isolated peptide for analysis. The population of bound peptide is estimated to have a complexity >2000, the majority of which are believed to be self-peptides. Therefore, the average molar amount of each individual peptide present after purification is 13 pmol divided by the population complexity. The utility of a large pool of purified peptides in which each individual species is present in exceedingly minute quantities is limited. At this point, the purified peptides can be fractionated by HPLC and the fractions assayed for reactivity with cloned CTLs. Tandem mass spectrometry can be used to sequence reactive fractions. However, the complexity of peptides in each fraction often exceeds the number of peptides that can be sequenced with the available material. Thus, although this method has been used successfully, the lack of data in the literature gleaned from this approach is testimony to the difficulty of its successful execution.

Knowledge of the primary sequence of MHC, or of known T cell epitopes, has not yielded a key to immunogenicity of such epitopes. Identification and screening of epitopes has also not been further facilitated by the determination of structural features of the MHC, e.g., using X-ray crystallography. These techniques, which in other systems provide for the rational design or identification of receptor agonists and antagonists, have not proven useful for identification of T cell epitopes.

Recombinant bacteriophage have been used to produce large libraries. Using the “phage method” (Scott and Smith (1990) Science 249:386-390; Cwirla et at. (1990) PNAS 87:6378-6382; Devlin et al. (1990) Science 249:404-406), very large libraries can be constructed (10⁶-10⁸ chemical entities). However, in these libraries it is difficult to dissociate a response due to the recombinant fusion protein from one due only to the peptide. Another approach uses primarily chemical methods, of which the Geysen method (Geysen et al. (1986) Molecular Immunology 23:709-715; and Geysen et al. (1987) J. Immunologic Method 102:259-274) and the recent method of Fodor et al. (1991) Science 251:767-773) are examples. Furka et al. (1988) 14th International Congress of Biochemistry, Volume 5. Abstract FR:013; Furka, (1991) Int. J. Peptide Protein Res. 37:487-493). Houghton (U.S. Pat. No. 4,631,211 issued December 1986) and Rutter et al. (U.S. Pat. No. 5,101,175, issued Apr. 23, 1991) describe methods to produce a mixture of peptides that can be tested as agonists or antagonists. However, these methods cannot be used alone to identify antigens because they provide either limited numbers of predesigned peptides, which require some advance predictions about the desired sequence, or in a large (and in the case of Rutter (U.S. Pat. No. 5,101,175, issued Apr. 23, 1991), supra chaotic and indiscriminate) mixture of peptides, leaving one no better off than with naturally purified MHC containing peptide epitopes.

A major advance in screening occurred with the development of synthetic libraries (Needels et al. (1993) PNAS 90:107004; Ohlmeyer et al. (1993) PNAS 90:10922-10926, Lam et al., International Patent Publication No. WO 92/00252, and the like that can be used to screen for receptor ligands.

The synthesis of indexed combinatorial peptide displays has been described (Ohlmeyer et al. (1993) Proc. Natl. Acad. Sci. USA 90:10922-26). It is possible to synthesize epitope-length peptides on Merrifield resin beads while cosynthesized inert molecular tags allow rapid and efficient decoding of the synthesis history of any unique bead via gas-phase chromatography (Ohlmeyer et al., supra) The efficiency of decoding is about 90 percent utilizing a single bead. Furthermore, it is not necessary to restrict assays to solid-phase interactions since photocleavable linkages allow controlled release of required amounts of peptide for solution-phase assays. This is important because it may not be possible for peptides to bind directly to surface-localized MHC class I molecules directly (in general, loading APCs with antigen occurs by internalizing the peptide and combining it with the MHC molecule as it assembles), and even if the APCs can bind directly to the beads, tight packing of the peptide on the surface of the beads may cause enough steric hindrance so as not to allow access of the MHC/peptide complex to the CTL T-cell receptors. Note that MHC/peptide complexes have a remarkable stability. A feature of most MHC/peptide complexes is their unusually slow dissociation kinetics, with a half-life in the range of several days. Most peptides (>90% of characterized human HLA-A epitopes) will bind with affinities of 2-50 nM.

Van der Zee (1989) Eur. J. Immunol. 19:4347 and coworkers have developed a powerful but limited strategy for identifying T-cell epitopes. Briefly, utilizing the “pepscan” technique, they were able to simultaneously synthesize several dozens of peptides on polyethylene rods arrayed in a 96-well microliter plate pattern. This is similar to an indexed library in that the position of each pin defines the synthesis history on it. Peptides were then chemically cleaved from the solid support and supplied to irradiated syngeneic thymocytes for antigen presentation. The cloned CTL line was then tested for reactivity in a proliferation assay monitored by ³H-thymidine incorporation. This type of analysis particularly suits a CTL stimulation assay since it can be automated using a microliter plate reader and employs relatively low levels of radiation. The procedure successfully identified a reactive epitope in a defined region of a 65 kDa mycobacterial heat shock protein with essentially no background. A second screen where the synthesized peptides had one alanine insertion per peptide at each position of the naturally occurring epitope identified an additional seven peptides with diminished yet detectable reactivity, underscoring the tolerances to substitutions in this assay. Additionally, screening peptides having a single deletion per peptide (derived from the natural epitope) yielded no reactive peptides, underscoring the specificity endowed by the presence of the nine residues in the naturally occurring epitope.

### EXPERIMENTAL

### I. Sources of Antigen Presenting Cells

The following methods are provided for the isolation and expansion of APC.

Suitable sources of antigen presenting cells include, but are not limited to, whole cells such as dendritic cells or macrophages; purified MHC class I molecule complexed to β2-microglobulin, and, as described below, foster antigen presenting cells.

Isolation of APC. The following is a brief description of various approaches for the isolation of APC such as dendritic cells. These approaches involve (1) isolating bone marrow precursor cells (CD34⁺) from blood and stimulating them to differentiate into DCs; or (2) collecting the precommitted DCs from peripheral blood. In the first approach, the patient must be treated with cytokines such as GM-CSF to boost the number of circulating CD34⁺ stem cells in the peripheral blood.

The second approach for isolating DCs is to collect the relatively large numbers of precommitted DCs already circulating in the blood. Previous techniques for preparing mature DCs from human peripheral blood have involved combinations of physical procedures such as metrizamide gradients and adherence/nonadherence steps (Freudenthal P S et al. (1990) PNAS 87:7698-7702); Percoll gradient separations (Mehta-Damani et al. (1994) J. Immunol. 153:996-1003); and fluorescence activated cell sorting techniques (Thomas R. et al. (1993) J. Immunol. 151:6840-52).

The preferred methods for isolation and culturing of DCs are described in Bender et al. (1996) J. Immun. Meth. 196: 121-135 and Romani et al. (1996) J. Immun. Meth. 196:137-151.

Foster Antigen Presenting Cells. The human cell line 174xCEM.T2, referred to as T2, contains a mutation in its antigen processing pathway that restricts the association of endogenous peptides with cell surface MHC class I molecules (Zweerink et al. (1993) J. Immunol. 150:1763-1771). This is due to a large homozygous deletion in the MHC class II region encompassing the genes TAP1, TAP2, LMP1, and LMP2 which are required for antigen presentation to MHC class I-restricted CD8⁺ CTLs. In effect, only “empty” MHC class I molecules are presented on the surface of these cells. Exogenous peptide added to the culture medium binds to these MHC molecules provided that the peptide contains the allele-specific binding motif. These T2 cells are referred to as “foster” APCs.

Retroviral infection or transfection of T2 cells with specific recombinant MHC alleles allows for redirection of the MHC restriction profile. Libraries tailored to the recombinant allele will be preferentially presented by them because the anchor residues will prevent efficient binding to the endogenous allele. In at least one case, the cell line 174×CEM.T2 was transfected with a mouse H-2Ld MHC allele which rendered the cells sensitive to an H-2Ld restricted CTL clone (Crumpacker et al. (1992) J. Immunol. 148:3004). This technique generates recombinant foster APCs specific for any MHC restricted CTL for which the variable chain of the MHC allele is cloned.

Several cases have demonstrated that transfection of non-professional APCs with allogenic MHC alleles aids greatly in the immunogenicity of the recombinant cell line (Leong et al. (1994) Int. J. Cancer 59:212-216; and Ostrand-Rosenberg et al. (1991) Int. J. Cancer Suppl. 6-61-68). That is to say, immunosensitivity is proportional to the level of expression of the MHC proteins. Thus, recombinant T2 cells are ideal APCs.

High level expression of MHC molecules makes the APC “more visible” to the CTLs. Expressing the MHC allele of interest in T2 cells using a powerful transcriptional promotor (e.g., the CMV promotor) results in a more reactive APC (most likely due to a higher concentration of reactive MHC-peptide complexes on the cell surface). Note that since only one type of MHC allele will be able to interact with a given library, the presence of or expression level of the endogenous allele will not compromise specificity if the library is designed to bind to the newly transfected allele.

Cell Free Antigen Presentation. Recently activation of CTLs has been achieved by incubating the antigenic peptide with purified MHC class I molecules complexed to β₂-microglobulin (Huang et al. (1994) Immunity 1:607-613). In this cell-free MHC/peptide binding assay, it was shown that the Kₘ and K_{d} approached physiologic levels, reaching equilibrium in 1-2 minutes. This eliminates the need for intact antigen presenting cells and may prove to be more efficient. There are numerous precedents in the literature utilizing intact, irradiated APCs to assay peptides in solution.

Antigen Painting. It has been demonstrated that glycosyl-phosphotidylinositol (GPI)-modified proteins possess the ability to reincorporate themselves back into cell membranes after purification. (Medof et al. J. Exp. Med. 160:1558-1578). Huang et al. Immunity 1:607-613 have exploited this property in order to create APCs of specific composition for the presentation of antigen to CTLs. They devised expression vectors for β2-microglobulin and the HLA-A2.1 allele. The proteins were expressed in Schneider S2 Drosophila melanogaster cells, known to support GPI-modification. After purification, the proteins could be incubated together with a purified antigenic peptide which resulted in a trimolecular complex capable of efficiently inserting itself into the membranes of autologous cells. In essence, these protein mixtures were used to “paint” the APC surface, conferring the ability to stimulate a CTL clone that was specific for the antigenic peptide. Cell coating was shown to occur rapidly and to be protein concentration dependent. This method of generating APCs bypasses the need for gene transfer into the APC and permits control of antigenic peptide densities at the cell surfaces. It is possible that this approach would allow the screening of a greater number of beads/well, since the problem of saturating the MHC binding sites can be managed by “painting” the APC at a higher MHC/peptide density.

### II. Isolation, Culturing and Expansion of CTLs

Various embodiments of this invention require the isolation of immune effector cells such as CTLs. The following describes methods for the isolation and culturing of CTLs.

Methods of isolating, culturing and expanding immune effector cells are known in the art. An effector cell population suitable for use in the present invention can be autogeneic or allogeneic, preferably autogeneic. When effector cells are allogeneic, preferably the cells are depleted of alloreactive cells before use. This can be accomplished by any known means, including, for example, by mixing the allogeneic effector cells and a recipient cell population and incubating them for a suitable time, then depleting CD69⁺ cells, or inactivating alloreactive cells, or inducing anergy in the alloreactive cell population.

The effector cell population can comprise unseparated cells, i.e., a mixed population, for example, a PBMC population, whole blood, and the like. The effector cell population also can be manipulated by positive selection based on expression of cell surface markers, negative selection based on expression of cell surface markers, stimulation with one or more antigens in vitro or in vivo, treatment with one or more biological modifiers in vitro or in vivo, subtractive stimulation with one or more antigens or biological modifiers, or a combination of any or all of these.

For example, effector cells can be isolated by leukapheresis, mechanical apheresis using a continuous flow cell separator. For example, lymphocytes and monocytes can be isolated from the buffy coat by any known method, including, but not limited to, separation over Ficoll-Hypaque™ gradient, separation over a Percoll gradient, or elutriation. The concentration of Ficoll-Hypaque™ can be adjusted to obtain the desired population, for example, a population enriched in T cells. Other methods based on affinity are known and can be used. These include, for example, fluorescence-activated cell sorting (FACS), cell adhesion, magnetic bead separation, and the like. Affinity-based methods may utilize antibodies, or portions thereof, which are specific for cell-surface markers and which are available from a variety of commercial sources, including, the American Type Culture Collection (Rockville, Md.). Affinity-based methods can alternatively utilize ligands or ligand analogs, of cell surface receptors.

The effector cell population can be subjected to one or more separation protocols based on the expression of cell surface markers. For example, the cells can be subjected to positive selection on the basis of expression of one or more cell surface polypeptides, including, but not limited to, “cluster of differentiation” cell surface markers such as CD2, CD3, CD4, CD8, TCR, CD45, CD45RO, CD45RA, CD11b, CD26, CD27, CD28, CD29; CD30, CD31, CD40L; other markers associated with lymphocyte activation, such as the lymphocyte activation gene 3 product (LAG3), signaling lymphocyte activation molecule (SLAM), T1/ST2; chemokine receptors such as CCR3, CCR4, CXCR3, CCR5; homing receptors such as CD62L, CD44, CLA, CD 146, α4β7, αEβ7; activation markers such as CD25, CD69 and OX40; and lipoglycans presented by CD1. The effector cell population can be subjected to negative selection for depletion of non-T cells and/or particular T cell subsets. Negative selection can be performed on the basis of cell surface expression of a variety of molecules, including, but not limited to, B cell markers such as CD19, and CD20; monocyte marker CD 14; the NK cell marker CD56.

An effector cell population can be manipulated by exposure, in vivo or in vitro, to one or more biological modifiers. Suitable biological modifiers include, but are not limited to, cytokines such as IL-2, IL-4, IL-10, TNF-α, IL-112, IFN-γ; non-specific modifiers such as phytohemagglutinin (PHA), phorbol esters such as phorbol myristate acetate (PMA), concanavalin-A, and ionomycin; antibodies specific for cell surface markers, such as anti-CD2, anti-CD3, anti-IL2 receptor, anti-CD28; chemokines, including, for example, lymphotactin. The biological modifiers can be native factors obtained from natural sources, factors produced by recombinant DNA technology, chemically synthesized polypeptides or other molecules, or any derivative having the functional activity of the native factor. If more than one biological modifier is used, the exposure can be simultaneous or sequential.

CTLs also can be isolated as described in WO 96/06929. Briefly, the method allows the T cells to be propagated in culture (i.e., the target T-cells) which are originally obtained from the subject. Alternatively, T cells can be obtained from persons other than the subject to be treated provided that the recipient and transferred cells are immunologically compatible. Typically, the cells are derived from tissue, bone marrow, fetal tissue, or peripheral blood. Preferably, the cells are derived from peripheral blood. If the T cells are derived from tissues, single cell suspensions should be prepared using a suitable medium or diluent.

Mononuclear cells containing the T lymphocytes are isolated from the heterogenous population according to any of the methods well known in the art, e.g., Ficoll-Hypaque gradient centriftigation, fluorescence-activated cell sorting (FACs), panning on monoclonal antibody coated plates. Alternatively, magnetic separation techniques can be used (separately or in combination with other separation methods) to obtain purified populations of cells for expansion according to the present invention. Antigen-specific T cell clones are isolated by standard culture techniques known in the art involving initial activation of antigen-specific T cell precursors by stimulation with antigen-presenting cells and subsequent cloning by limiting dilution cultures using techniques known in the art, such as those described in Riddell and Greenberg (1990) J. Immunol. Meth. 128:189-201, and Riddell et al. (1991) J. Immunol. 146:2795-1804.

The T cell clones isolated in microwells in limiting dilution cultures typically have expanded from a single cell to 2×10⁴ to 5×10⁵ cells after 14 days. At this time individual clones are placed in appropriate culture media in plastic culture vessels with disproportionately large numbers of feeder cells which provide co-stimulatory functions, and, preferably, anti-CD3 monoclonal antibody to provide T cell receptor stimulation. This initial phase of rapid expansion when the clone is transferred from a microwell is generally carried out in a culture vessel, the size of which depends upon the number of target cells, and which may typically be a 25 cm² flask. The size of the culture vessel used for subsequent cycles of T cell expansion depends on the starting number of T cells and the number of cells needed (usually for therapeutic use).

### III. Identification of Sequence Motifs in Antigens Recognized by Immune Effector Cells

In one aspect, the methods of this invention utilize SPHERE technology, as described below. SPHERE is utilized to identify one or more genes encoding antigens recognized by immune effector cells. The SPHERE method requires the steps of:
(i) creating a library of molecules attached to solid phase supports by a releasable linker, wherein each solid phase support is attached to a single species of molecule;
(ii) providing a source of antigen presenting cells having the same MHC restriction as the immune effector cells;
(iii) cleaving at least a portion of the releasable linker so as to release at least a portion of the molecule(s);
(iv) contacting the immune effector cells with the library and the antigen presenting source under conditions that favor the immune effector cells recognizing one or more of the molecule(s) that have been released from the solid phase supports as in step (iii) above;
(v) evalulating whether the immune effector cells recognize one or more molecule(s) present in the library;
(vi) determining the structure of the molecule(s) recognized by the immune effector cells; and
(vii) identifying a sequence motif common to the molecule(s) recognized by the immune effector cells;

The solid phase support is selected from the group consisting of polystyrene resin, poly(dimethylacryl)amide-grafted styrene-co-divinylbenzene resin, polyamide resin, polystyrene resin grafted with polyethylene glycol, and polydimethylacrylamide resin.

A modification of the SPHERE method provides a releasable linker that releases upon exposure to an acid, a base, a nucleophile, an electrophile, light, an oxidizing agent, a reducing agent or an enzyme.

The exact sequence of the steps may be modified as appropriate.

Library Complexities. In one embodiment of the invention, the molecules attached to the solid support are peptides. However, to synthesize a peptide library of completely degenerate 8-mers would result in an intractable complexity on the order of 10¹⁰. Fortunately, several invariant amino acids have been identified which serve as anchor residues on the peptide for binding to the MHC molecule. These anchor positions are different for each subclass of MHC class I molecules, but in each case there are two or three dominant anchor positions (Falk et al. (1991) Nature 351:290-296). Interestingly, a single MHC molecule is capable of binding many different peptides as long as the anchoring amino acids are present or at least conserved.

Inclusion of two invariant positions in each peptide reduces the complexity by a factor of 400, resulting in a complexity of 20⁶ or 6.4×10⁷ for an octamer library.

This complexity can be further reduced by making some calculated assumptions. For epitopes with high MHC binding affinity (i.e., optimal anchor residues), conservative substitutions at non-anchor positions do not interfere with recognition by CTL TCRs. That is to say, these conservatively substituted peptides are seen qualitatively as a single entity to the appropriate TCR rather than as individual entities. For example, a human HLA-B27 restricted CTL clone specific for the HIV gag p24 protein (residues 263-271) could not distinguish between valine and isoleucine at position five, or methionine and leucine at position six. However a nonconservative substitution of glutamic acid for glycine at position seven was no longer recognized (Johnson et al. J. Immunol. 147:1512-1521). The limits of TCR tolerance to conservative substitutions are not known and cannot be accurately assessed until saturating profiles of reactive epitopes are empirically determined for a statistically significant number of CTLs. Synthesizing a library in which one or two representatives of each class of amino acids are coupled at each position (for example leucine and methionine but not isoleucine and valine in the case of hydrophobic amino acids) can reduce the library to a manageable complexity. Arguably, a given tumor will elicit CTL clones specific for more than one individual tumor-specific epitope. Screening a less degenerate library using the CTL clones (in batch) in the proliferation assay can maintain statistical confidence in success even though the complexity is reduced. The trade-off is that a saturating profile of reactive epitopes may not be achieved. However, screening a library in which all non-anchor positions are completely degenerate, though enormously complex, results in the same signal to noise ratio as an incompletely degenerate library. The “signal” refers to beads that will correctly register as reactive in the assay and the “noise” is the sum total of all non-specific reactive species. In theory this larger library could provide a saturating profile of reactive peptides, however, it will not because it cannot be practicably screened in its entirety. The advantage of using the more complex library is that all possible epitopes will be represented. What is not readily obvious is that the chances of identifying a reactive epitope from the more complex library is no greater than those of the less complex library when the same number of beads are assayed. That is to say, the signal to noise ratio, and thus the probability of successful screening, is the same for both libraries.

The composition of first (primary) screen MHC allele-specific libraries must (1) have a high affinity for the particular MHC allele (i.e., an effective agretope) and (2) provide a sufficiently diverse repertoire of motifs to interact with TCRs (i.e., the epitopes) in order that the natural epitope and/or its reactive derivatives to be represented to the extent that at least one positive can be detected by screening a manageable number of beads. Until the limits of TCR promiscuity are known, determination of the number of beads that must be screened cannot be accurately calculated. However, the few reports in the literature that address this issue are encouraging. If this small sample size is a representative cross section of general TCR behavior, then it is predicted that the beads necessary to generate 10⁵, different peptides (e.g., a number calculated from Poisson distribution statistics) will suffice to at least identify a reactive derivative. Once identified, even if it is only weakly reactive, the secondary screen will provide for enumeration of the complete spectrum of reactive derivatives. In most cases the secondary screen will involve far less than 10⁵ different sequences.

Thus, one particular advantage of the invention is that it greatly reduces the complexity of the library screening procedures.

To a first approximation it can be assumed that conservative substitutions will be tolerated and will perhaps, more often than not, result in partial loss of activity. (Hobohm and Meyerhans (1993) Eur. J. Immunol. 23:1271-1276), have developed an algorithm to detect MHC binding motifs which correlates well with known, empirically determined, MHC binding motifs. The best results were obtained using a similarity matrix based on the physicochemical properties of the amino acids (Taylor (1986) J. Theor. Biol. 119:205) rather than the Dayhoff matrix PAM250 (Dayhoff et al. (1978) ATLAS OF PROTEIN SEQUENCE AND STRUCTURE Vol. 5. Suppl. 3:345), the three inside matrices ‘inside alpha’. inside beta’ and ‘inside other’ (Luethy et al. (1991) Proteins 10:229) or the substitution probability table for inaccessible residues (Overington et al. (1992) Protein Sci. 1:216). Thus, the physicochemical properties matrix seems to embody the criteria important to MHC-peptide-TCR interactions.

TABLE 1
Physiochemical Properties Matrix

| Amino Acid | Hydrophobic | Positive | Negative | Aliphatic | Aromatic |
|---|---|---|---|---|---|
| A | 1 | 0 | 0 | 0 | 0 |
| C | 1 | 0 | 0 | 0 | 0 |
| D | 0 | 0 | 1 | 0 | 0 |
| E | 0 | 0 | 1 | 0 | 0 |
| F | 1 | 0 | 0 | 0 | 1 |
| G | 1 | 0 | 0 | 0 | 0 |
| H | 0 | 1 | 0 | 0 | 1 |
| 1 | 1 | 0 | 0 | 1 | 0 |
| K | 0 | 1 | 0 | 0 | 0 |
| L | 1 | 0 | 0 | 1 | 0 |
| M | 1 | 0 | 0 | 0 | 0 |
| N | 0 | 0 | 0 | 0 | 0 |
| P | 0 | 0 | 0 | 0 | 0 |
| Q | 0 | 0 | 0 | 0 | 0 |
| R | 0 | 1 | 0 | 0 | 0 |
| S | 0 | 0 | 0 | 0 | 0 |
| T | 1 | 0 | 0 | 0 | 0 |
| V | 1 | 0 | 0 | 1 | 0 |
| W | 1 | 0 | 0 | 1 | 0 |
| Y | 1 | 0 | 0 | 1 | 0 |
| TOTAL | 13 | 3 | 2 | 3 | 4 |

“1” indicates the amino acid possesses the property; “0” indicates it does not.

Using these data, a theoretical reactive peptide substitution profile can be generated. For example, if a residue in an epitope is negatively charged, assume that D or E will work to some degree; if a residue is positively charged, H, K or R will work. So if the epitope is YLKDQQLL (anchor residues shown in bold), there are 70,304 conservatively substituted derivatives. Therefore, for a library with a complexity of 20⁶, less than 1000 beads need to be screened in order to identify a derivative. This calculation assumes that all possible combinations of conservative substitutions retain some activity. A more likely scenario is that only a fraction of these will be detectably active. Screening 10⁶, beads (i.e., ten 96-well plates at 1000 beads per well) would be successful if only 0.09% of these derivatives demonstrate detectable activity. Additionally, the simultaneous screening of multiple CTLs contributes greatly to the chances of success. It will be possible to further refine these calculations when a statistically significant number of complete spectrums of TCR tolerances are empirically determined, e.g., using the methods of the present invention.

Multiple substitutions within an epitope have been observed to be fully functional by way of a redistribution of the relative contributions of each residue to the trimolecular complex, thus challenging the static definition of agretope and epitope as MHC and TCR binding sites (Reddehase and Koszinowski (1991) Eur. J. Immunol. 21:1697-1701; Boyer et al. Eur. J. Immunol. 20:2145-2148). Also, a mouse CTL epitope derived from pigeon cytochrome C was shown to have complete tolerance to at least 7 substitutions (A, V, L, N, Q, K, M) at an internal residue presumed to be part of the epitope rather than the agretope (Ogasawara et al. (1990) Int. Immunol. 2:219-224). The sensitivity of the primary screen can be tuned by adjusting the number of beads and/or the number of CTL lines screened. This analysis demonstrates the feasibility of utilizing libraries with complexities on the order of 10⁷.

Compositions of HLA Allele-Specific Oligopeptide Libraries

Table 2 gives the optimal library compositions for various MHC alleles based on the literature and the above considerations:

TABLE 2

| | SEQ ID | | |
|---|---|---|---|
| MHC Allele | NO | Composition | Reference¹ |
| HLA A2.1² | 1 | LXXXXXXV | Henderson et al., 1992 |
| HLA B27³ | 2 | RXXXXXX+ | Jardetzky et al., 1991 |
| HLA A1 | 3 | X(D,E)XXXXXX(F,K,Y) | Hobohm et al., 1993 |
| HLA B14 | 4 | RXXXXXXL | Hobohm et al. . 1993 |
| HLA B8 | 5 | X(K,R)XXXXX(L,I) | Hobohm et al.. 1993 |
| HLA A3 | 6 | (ML)XXXXXXK | DiBrino et al., 1993 |
| HLA B44 | 7 | EXXXXXX(Y,F) | DiBrino et al., 1995 |
| HLA B7-1-Sm⁴ | 8 | XPXXXXX(F,H,W,Y) | Sidney et al., 1995 |
| HLA All | 9 | (LI)XXXXX(H,K) | Hobohm et al., 1993 |

Anchor residues are shown in bold. “X” indicates a completely degenerate position.
indicates a positively charged residue (i.e., H,K,R).
¹DiBrino et al., 1993, J. Immunol., 151: 5930-5935; DiBrino et at., 1995, 34: 10130-10138; Fruci et at., 1983, Hum. Immunol. 38: 187; Henderson et al., 1992, Science, 255: 1264; Hobohm et al., 1993, Eur. J. Immunol. 23: 1271; Jardetzky, 1991, Nature, 353: 326; Sidney et at., 1995, J. Immunol. 154: 247.
²Crossreactive with HLA A24, A26, A28, A29 (Fruci et al., 1993, Hum. Immunol. 38: 187-192)
³Crossreactive with HLA A31 (Fruci et at., 1993, supra)
⁴HLA B7-like supermotif, crossreactive with HLA- B*0701, −B*0801, −B*2705, −B*3501-03, −B*5401, −Cw*0401, −Cw*0602, −Cw*0702

Solid Phase Combinatorial Libraries. In addition to using naturally occuring peptides, any one of the many combinatorial library technologies described to date can be employed in the practice of the present invention, including but not limited to synthetic combinatorial peptide or molecule libraries (Needels et al. (1993) Proc. Natl. Acad. Sci. USA 90:10700-4; Ohlmeyer et al. (1993) Proc. Natl. Acad. Sci. USA 90:10922-10926; Lam et al., International Patent Publication No. WO 92/00252; Kocis et al., International Patent Publication No. WO 94/28028, published Dec. 8, 1994), and the like can be used to screen for CTL epitopes according to the present invention.

In a preferred aspect, the invention employs the solid phase library technique described by Ohlmeyer et al. (1993). Halogen substituted benzenes linked to tag-liner tert-butyl esters constitute the inert molecular tags that encode the sequence of the unique peptide co-synthesized on any given bead in the library. A brief description of the tag synthesis, peptide synthesis, and encoding/decoding strategy is presented below.

The molecular tags used as encoding molecules are precipitated from dimethylformamide (DMF) containing 8-bromo-1-octanol and 2,4,6-tricholorophenol by the addition of cesium carbonate. The solution is then heated to 80° C. for 2 hours, washed with 0.5M NaOH, 1M HCl and finally H₂O, at which point the organic phase is evaporated. The resulting tag alcohol is a colorless oil.

The tag alcohol is then added to a 2 M solution of phosgene (in toluene) to produce a crude chloroformate. After evaporation of the solvent, the compound is dissolved in CH₂Cl₂ and pyridine and incubated with tert-Butyl-4-(hydroxymethyl)-3-nitrobenzoate. The resulting tag-linker tert-butyl ester is isolated from the organic phase and purified by chromatography with the product being a clear oil. To generate unique tags, halogen-substituted benzene compounds are reacted with the electrophoric tag. Each derivative has a different gas chromatography retention time. This property confers the ability to encode the unique synthesis history of individual beads. Electron capture capillary gas chromatography can selectively detect the tags at levels less than 1 pmol.

Preferably, the peptides are synthesized on Merrified resin beads (or other suitable resin) such that the peptides are linked by photocleavable crossovers by a typical split-synthesis method. As each amino acid is added, a corresponding mixture of acyl carbonate-activated linker tag acids is co-ligated, but with a linker which is not photocleavable. This allows release of the peptide with retention of the coding molecules during the screening procedure. The combination of tag molecules added at each step corresponds to the specific amino acid residue added in that step, thus serving as a record of the synthetic history of any given bead.

When a bead of interest is identified, the sequence of the peptide that was synthesized on it can be deduced by the following method. The bead is loaded into a Pyrex capillary tube and washed with DMF. It is then suspended in 1 μl DMF and sealed in the capillary tube and irradiated to release the tag alcohols. The capillary tube is then opened and the tag alcohols are trimethlsilyted with bis(trimethylsilyt)acetamide. The solution above the bead is then injected into an electron capture, capillary gas chromatograph for analysis. The resulting profile of tag elution on the gas chromatogram allows the amino acid sequence of the co-synthesized peptide to be directly determined.

Peptide libraries can include unnatural amino acids. Thus, peptides of the invention may comprise D-amino acids, a combination of D- and L-amino acids, and various “designer” amino acids (e.g., β-methyl amino acids, Ca-methyl amino acids, and Nα-methyl amino acids, etc.) to convey special properties to peptides in the library. Additionally, by assigning specific amino acids at specific coupling steps, peptide libraries with α-helices, β turns, β sheets, γ-turns, and cyclic peptides can be generated. Generally, it is believed that α-helical secondary structure or random secondary structure is preferred.

In a specific aspect of the invention, the peptides of a library may comprise a special amino acid at the C-terminus which incorporates either a CO₂H or CONH₂ side chain to simulate a free glycine or a glycine-amide group. Another way to consider this special residue would be as a D or L amino acid analog with a side chain consisting of the linker or bound to the bead. In one embodiment, the pseudo-free C-terminal residue may be of the D or the L optical configuration; in another embodiment, a racemic mixture of D and L-isomers may be used.

In an additional embodiment, pyroglutamate may be included as the N-terminal residue of the peptides of the library. Although pyroglutamate is not amenable to sequence by Edman degradation, identification of the peptide sequence can be accomplished by a coded library strategy, or by limiting substitution to only 50% of the peptides on a given bead with N-terminal pyroglutamate, thus leaving enough non-pyroglutamate peptide on the bead for direct sequencing. One of ordinary skill would readily recognize that this technique could be used for sequencing of any peptide that incorporates a residue resistant to Edman degradation at the N-terminus. Specific activity of a peptide that comprises a blocked N-terminal group. e.g., pyroglutamate, when the particular N-terminal group is present in 50% of the peptides, would readily be demonstrated by comparing activity of a completely (100%) blocked peptide with a non-blocked (0%) peptide.

In a further embodiment, subunits of peptides that confer useful chemical and structural properties will be chosen. For example, peptides comprising D-amino acids will be resistant to L-amino acid-specific proteases in vivo. In addition, the present invention envisions preparing libraries of peptides that have more well defined structural properties, and the use of peptidomimetics, and peptidomimetic bonds, such as ester bonds, to prepare libraries with novel properties. In another embodiment, a peptide library may be generated that incorporates a reduced peptide bond, i.e., R₁—CH₂—NH—R₂, where R₁ and R₂ are amino acid residues or sequences. A reduced peptide bond may be introduced as a dipeptide subunit. Such a molecule would be resistant to peptide bond hydrolysis, e.g., protease activity. Such libraries would provide ligands with unique function and activity, such as extended half-lives in vivo due to resistance to metabolic breakdown, or protease activity. Furthermore, it is well known that in certain systems constrained peptides show enhanced functional activity (Hruby, (1982) Life Sciences 31:189-199; Hruby et at. (1990) Biochem J. 268:249-262); the present invention provides a method to produce a constrained peptide that incorporates random sequences at all other positions.

Non-classical amino acids that induce conformational constraints. The following nonclassical amino acids may be incorporated in the peptide library in order to introduce particular conformational motifs: 1,2,3,4-tetrahydroisoquinoline-3-carboxylate (Kazmierski et al. (1991) J. Am. Chem. Soc. 113:2275-2283); (2S,3S)-methyl-phenylalanine, (2S,3R)methyl-phenylalanine, (2R,3S)-methyl-phenylalanine and (2R,3R)-methyl-phenylalanine (Kazmierski and Hruby, 1991, Tetrahedron Lett.); 2-aminotetrahydronaphthalene-2carboxylic acid (Landis, 1989, Ph.D. Thesis, University of Arizona); hydroxy-1,2,3,4-tetrahydroisoquinoline-3-carboxylate (Miyake et al. (1989) J. Takeda Res. Labs. 43:53-76), β-carboline (D and L) (Kazmierski, 1988, Ph.D. Thesis, University of Arizona); HIC (histidine isoquinoline carboxylic acid) (Zechel et al. (1991) Int. J. Pep. Protein Res. 43); and HIC (histidine cyclic urea).

The following amino acid analogs and peptidomimetics may be incorporated into a library to induce or favor specific secondary structures: LL-Acp (LL-3-amino-2-propenidone-6-carboxylic acid), a β-turn inducing dipeptide analog (Kemp et al. (1985) J. Org. Chem. 50:5834-5838); β-sheet inducing analogs (Kemp et al. (1988) Tetrahedron Lett. 0.29:5081-5082); β-turn inducing analogs (Kemp et al. (1988) Tetrahedron Lett. 29:5057-5060); ∝-helix inducing analogs (Kemp et al. (1988) Tetrahedron Lett. 29:4935-4938); γ-turn inducing analogs (Kemp et al. (1989) J. Org. Chem. 54:109:115); and analogs provided by the following references: Nagai and Sato (1985) Tetrahedron Lett. 26:647-650; DiMaio et al. (1989) J. Chem. Soc. Perkin Trans. p. 1687; also a Gly-Ala turn analog (Kahn et, al (1989) Tetrahedron Lett 30:2317); amide bond isostere (Jones et al. (1988) Tetrahedron Lett. 29:3853-3856); tretrazol (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am. Chem. Sci. 112:323-333 and Garvey et al. (1990) J. Org. Chem. 56:436. Conformationally restricted mimetics of beta turns and beta bulges, and peptides containing them, are described in U.S. Pat. No. 5,440,013, issued Aug. 8, 1995 to Kahn.

Determination of the sequence of peptides that incorporate such non-classical amino acids is readily accomplished by the use of a coded library. Alternatively, a combination of initial Edman degradation followed by amino acid analysis of the residual chain can be used to determine the structure of a peptide with desired activity. Mass spectral analysis may be employed.

Solid phase supports and linkers. A solid phase support for use in the present invention will be inert to the reaction conditions for synthesis. A solid phase support for use in the present invention must have reactive groups in order to attach a monomer subunit, or for attaching a linker or handle which can serve as the initial binding point for a monomer subunit. In one embodiment, the solid phase support may be suitable for in vivo use, i.e., it may serve as a carrier for or support for direct applications of the library (e.g., TentaGel, Rapp Polymere, Tubingen, Germany).

Preferably, the solid phase supports of the invention also comprise a cleavable linker. As used herein, a cleavable linker refers to any molecule that provides spatial distance between the support and the peptide to be synthesized, and which can be cleaved to provide for release of the peptide from the support into solution. Linkers can be covalently attached on the solid phase support prior to coupling with a N^{α}-Boc or N^{α}-Fmoc or otherwise appropriately protected amino acids. Various linkers can be used to attach the oligomer to solid phase support.

Examples of spacer linkers include aminobutyric acid, aminocaproic acid, 7-aminoheptanoic acid, and 8-aminocaprylic acid. Fmoc-aminocaproic acid is commercially available from Bachem Biochem, and is the preferred embodiment. In a further embodiment, linkers can additionally comprise one or more β-alanines as spacers.

In addition, the solid-support could be modified to meet specific requirements for the particular purpose of bioassay or detection. Modification of solid phase support may be made by incorporation of a specific linker. For example, modified solid phase support could be made acid-sensitive, base-sensitive, nucleophilic-sensitive, electrophilic sensitive, photosensitive, oxidation sensitive or reduction sensitive.

In addition to the linkers described above, selectively cleavable linkers may be employed. For example, an ultraviolet light sensitive linker, ONb, can be used (see Barany and Albericio, 1985, J. Am. Chem. Soc. 107:4936-4942). Other cleavable linkers require hydrogenolysis or photolysis. Examples of photosensitive (photocleavable) linkers are found in Wang (1976) J. Org. Chem. 41:32-58, Hammer et al. (1990) Int. J. Pept. Protein Res. 36:31-45, and Kreib-Cordonier et al. (1990) in PEPTIDES—CHEMISTRY. STRUCTURE AND BIOLOGY, Rivier and Marshall, eds., pp. 895-897. Landen (1977) Methods Enzym. 47:145-149 used aqueous formic acid to cleave Asp-Pro bonds; this approach has been used to characterize T-cell determinants in conjunction with the Geysen pin synthesis method (Van der Zee et al., (1989) Eur. J. Immunol. 191:43-47). Other potential linker groups cleavable under basic conditions include those based on p-(hydroxylmethyl) benzoic acid (Atherton et al., (1981) J. Chem. Soc. Perkin I:538-546) and hydroxyacetic acid (Baleaux et al., (1986) Int. J. Pept. Protein Res. 28:22-28). Geysen et al. (1990) J. Immunol. Methods 134:23-33) reported peptide cleavage by a diketopiperazine mechanism. An enzyme may specifically cleave a linker that comprises a sequence that is sensitive or a substrate for enzyme cleavage, e.g., protease cleavage of a peptide.

In certain instances, one may derivatize 10-90% of the resin by substitution with the cleavable linker, and the remaining 90-10% substituted with a noncleavable linker to ensure that after cleavage of linker enough peptide will remain for sequencing. Preferably, however, a cleavable linker is used in combination with a coded library strategy. Combinations of cleavable linkers can also be used to allow sequential cleaving from a single bead.

Expression of Signal Accessory Molecules. Research accumulated over the past several years has demonstrated convincingly that resting T cells require at least two signals for induction of cytokine gene expression and proliferation (Schwartz, R. H. (1990) Science 248:1349-1356; Jenkins, M. K. (1992) Immunol. Today 13:69-73). One signal, the one that confers specificity can be produced by interaction of the TCR/CD3 complex with an appropriate MHC/peptide complex. The second signal is not antigen specific and is termed the “costimulatory” signal. This signal was originally defined as an activity provided by bone-marrow-derived accessory cells such as macrophages and dendritic cells, the so called “professional” APCs. Several molecules have been shown to enhance costimulatory activity. These are heat stable antigen (HSA) (Liu, Y. et al. (1992) J. Exp. Med. 175:437-445), chondroitin sulfate-modified MHC invariant chain (Ii-CS) (Naujokas, M. F. et al. (1993) Cell 74:257-268), intracellular adhesion molecule 1 (ICAM-1) (Van Seventer, G. A. (1990) J. Immunol. 144:45794586), B7-1, and B7-2/B70 (Schwartz, R. H. (1992) Cell 71:1065-1068). These molecules each appear to assist co-stimulation by interacting with their cognate ligands on the T cells. The stimulatory ability of APCs prepared by either method described above may be enhanced by the introduction of genes that have been shown to provide co-stimulatory signals. The benefits of such enhancement may include the need for lower peptide concentrations and an improved signal-to-noise ratio in large-scale screens.

Method of Detection and Identification of Reactive Oligopeptides

In general, a screening assay of the invention will involve the steps of contacting antigen presentation means, e.g., antigen presenting cells, with a limited number of individual beads from a library (either a primary library with the greatest degeneracy of structure types, or a secondary library with degenerate residues of limited chemical types), cleaving peptides from the library to bind to MHC molecules of the antigen presentation means, and simultaneously or subsequently contacting the MHC-peptide complexes to the immune effector cells, preferably CTLs, of interest.

Cytolytic activity of the cells can be measured in various ways, including, but not limited to, tritiated thymidine incorporation (indicative of DNA synthesis), and examination of the population for growth or proliferation, e.g., by identification of colonies. (See, e.g., WO 94/21287). In another embodiment, the tetrazolium salt MTT (3-(4,5-dimethyl-thazol-2-yl)-2,5-diphenyl tetrazolium bromide) may be added (Mossman (1983) J. Immunol. Methods 65:55-63; Niks and Otto (1990) J. Immunol. Methods 130:140-151). Succinate dehydrogenase, found in mitochondria of viable cells, converts the MTT to formazan blue. Thus, concentrated blue: color would indicate metabolically active cells. In yet another embodiment, incorporation of radiolabel, e.g., tritiated thymidine, may be assayed to indicate proliferation of cells. Similarly, protein synthesis may be shown by incorporation of ³⁵S-methionine. In still another embodiment, cytotoxicity and cell killing assays, such as the classical chromium release assay, may be employed to evaluate epitope-specific CTL activation. Other suitable assays will be known to those of skill in the art.

The specific details of the preferred method are as follows: the library being screened is selected to match (in terms of anchor residues) the MHC allele expressed by the tumor. The library is plated out in 96-well flat bottomed plates at a density of approximately 100 to about 5000, preferably 1000-5000, beads per well (i.e., 20-1000, preferably 20-100, plates for a library of 10⁷ peptides). Each bead, on average, contains 200 pmol of peptide, so release of 50% of the product yields 100 pmol of solubuized peptide per well. The final assay volume can be 100-200 μl, so 100 pmol results in a final concentration of 0.5-1 μM. At this concentration, the peptide will spontaneously bind to the MHC molecules on the surface of the foster APCs. For the purposes of conserving peptide, the reaction may be supplemented with free β₂-micro globulin, allowing the peptides to bind at concentrations of 0.01-0.1 μM (Rock et al. (1992) Proc. Natl. Acad. Sci. USA 89:8918-8922). Note that an extremely small number of MHC molecules need to be occupied with the peptide in order to elicit a response because a single complex can serially engage and trigger up to approximately 200 TCRs (Valitutti et al. (1995) Nature 375:148-151). The method used for the partial release depends on the type of cleavable linker used. At this point, in order to be conservative with CTLs, wells are further pooled so that each well contains ˜10,000 peptides. In this way, 10⁷ beads can be screened in ten 96-well plates. The master plates containing the beads and 1° daughter plates containing unused peptide can be stored at −70° C. for reuse. The 2° daughter plates containing the pooled peptides are ready for screening. These plates may be prepared in advance and stored under the appropriate conditions so that the library is ready whenever the CTLs are at peak activity.

Strategy 1. The supernatant from each well is distributed to replica plates and 1-2×10³ irradiated (1500 rads) foster APCs (expressing the proper MHC allele) are added to each well. Next, the cloned CTLs are added to a total of 10³-10⁴ cells representing equal amounts of 10-20 different clones of the same MHC restriction such that the total final volume per well is 200 μl and the plates are incubated in a humidified CO₂ incubator for 4 days at 37° C. Each well is then pulsed with 18.5 kBq of [³H] dThd to measure CTL proliferation. 16 hours later, the radioactivity incorporated into the DNA of mitotically active CTLs is assayed by scintillation counting (Estaquier et at., 1994, The mixotope: a combinatorial peptide library as a T cell and B cell Immunogen., Eur. J. Immunol., 24:2789-2795). The magnitude of the proliferative response may serve as a preliminary screen for crossreacting epitopes. The greater the response the more likely it is that more than one CTL clone was stimulated. While all reactive peptides are of interest, the most efficacious vaccine candidates will be those that crossreact with CTLs derived from independent donors and which are restricted by the most common MHC alleles. Note that identification of epitopes containing the HLA B7-like supermotif (see TABLE 2) would be of great value as vaccine candidates since it will bind to many HLA B alleles which are represented in over 40% of individuals from all major ethnic groups (Sidney et at. (1995) J. Immunol. 154:247-259).

Strategy 2. Alternatively, the first step is to administer ⁵¹Cr-labeled T2 cells to the wells of the 2° daughter plates, followed by the addition of the CTLs. After 4 hours the released ⁵¹Cr is measured in the standard manner. When a positive well is identified, the 10 wells from the 1° daughter plate that correspond to that well are similarly assayed. At this point, the epitope search is narrowed down to the beads in a single well on one of the master plates.

Wells that register positive will be further analyzed as follows: The beads that correspond to the positive well are manually distributed (1 per well) to new plates and the remaining peptide is released from each. These plates are assayed as before, and in this way the reactive bead(s) are unambiguously isolated. The positive bead(s) can be rapidly and efficiently decoded since the molecular tags that encode the bead's synthesis history has remained on the bead (coupled with a non-photocleavable crosslinker). For example, analysis of the bead(s) by electron capture capillary gas chromatography inunediately reveals the peptide sequence that was synthesized on that bead (Ohlmeyer et al. (1993) supra). Thus the unambiguous identification of an epitope can be achieved in approximately ten days using the ³H-thymidine incorporation assay and in as few as two days if a ⁵¹Cr-release assay is used.

In another embodiment, application of the library beads to the surface of freshly poured top agar in a standard tissue culture plate, followed by release of a portion of the peptide, will result in a three dimensional concentration gradient of eluted peptide around each bead. Antigen presenting cells could be present in the top agar or applied to the surface after peptide release. Next, the CTL(s) of interest are plated over the top agar/peptide/APCs, followed by incubation at 37° C. for 4-12 hours. Reactive beads may be detected by the formation of plaques, where the size of the plaque indicates the magnitude of the response. Positive beads can then be taken from the plate, washed, and sequenced. This assay requires very little manual manipulation of the beads and the entire library can be screened simultaneously (in one step) in as little as four hours. Furthermore, the beads can be recovered, washed in 6M guanidiium, and reused.

In another embodiment, the described method for the identification of CD8⁺ MHC Class I restricted CTL epitopes can be applied to the identification of CD4⁺ MHC Class II restricted helper T-cell (Th) eptitopes. In this case, MHC Class II allele-specific libraries are synthesized such that haplotype-specific anchor residues are represented at the appropriate positions. MHC Class II agretopic motifs have been identified for the common alleles (Raunensee (1995) Curr. Opin. Immunol. 7:85-96; Altuvia et al. (1994) Mol. Immunol. 24:375-379, Reay et al. (1994) J. Immunol. 152:3946-3957; Verreck et al. (1994) Eur. J. Immunol. 24:375-379; Sinigaglia and Hammer (1994) Curr. Opin. Immunol. 6:52-56; Rotzschke and Falk (1994) Curr. Opin. Immunol. 6:45-51). The overall length of the peptides will be 12-20 amino acid residues, and previously described methods may be employed to limit library complexity. The screening process is identical to that described for MHC Class I-associated epitopes except that B lymphoblastoid cell lines (B-LCL) are used for antigen presentation rather than T2 cells. In a preferred aspect, previously characterized B-LCLs that are defective in antigen processing (Mellin's et al. (1991) J. Exp. Med. 174:1607-+1615); thus allowing specific presentation of exogenously added antigen, are employed. The libraries are screened for reactivity with isolated CD4⁺ MHC Class II allele-specific Th cells. Reactivity may be measured by ³H-thymidine incorporation according to the method of Mellins et al. supra, or by any of the methods previously described for MHC Class I-associated epitope screening.

The final step is to synthesize a library (of minimal complexity) which represents conservatively substituted derivatives of the identified epitope in order to isolate the most efficient cytolytic stimulator of the CTL clone(s). This second library can be used to sort out which of the CTLs originally assayed are responding to the peptide as well as identify the most efficacious peptide derivative. Note that the naturally occurring epitope may not be the most efficient stimulator. If one organizes the amino acids into chemically related groups, composition and complexity of the derivative libraries can be readily calculated. The amino acid groupings chosen for the design of secondary screen libraries is shown in TABLE 3. This table provides for the design of derivative libraries that are diverse yet easily manageable in terms of size. The amino acids can be loosely grouped according to their physicochemical properties as follows:

TABLE 3

| Amino Acid Group | Class |
|---|---|
| A, V, L, I, P, F, W, M | NONPOLAR SIDE CHAINS |
| G, S, T, C, Y, N, Q | UNCHARGED POLAR SIDE CHAINS |
| D, E | NEGATIVELY CHARGED SIDE CHAINS |
| K, R, H | POSITIVELY CHARGED SIDE CHAINS |

For example a derivative library for the sequence YLKDQQLL (SEQ ID NO: 10), actually a known HLA-B8 epitope, would look like this:

| | |
|---|---|
| X₁X₂KX₃X₄X₅X₆L | (SEQ ID NO: 11) |

X₁=G,S,T,C,Y,N,Q
X₂=A,V,L,I,P,F,W,M
X₃=D,E,
X₄=G,S,T,C,Y,N,Q,
X₅=G,S,T,C,Y,N,Q
X₆=A,V,L,I,P,F,W,M
   where the HLA-B8 anchor residues are shown in bold type. This library would have a complexity of 43,904. Determining the complete spectrum of reactive derivatives will provide information as to the extent and limits of TCR promiscuity and allow the design of better primary screen libraries.

### IV. Identification of Nucleotides Expressed by the Cells Recognized by the Immune Effector Cells

In one embodiment, the present invention utilizes SAGE. Using SAGE, sequence tags corresponding to expressed genes can be identified and analyzed.

The sequence tags corresponding to the expressed genes are prepared essentially as follows. First, a sample containing the genes of interest is provided. Suitable sources of samples include cells, tissue, cellular extracts or the like. Preferably, the sample is taken from an individual having a particular disease state of interest or at a particular stage in its development. Complementary DNA (cDNA) is then isolated from the sample, for example using methods known to those skilled in the art. As described for example in CURRENT PROTOCOLS IN MOLECULAR BIOLOGY, supra. In one embodiment, the cDNA is synthesized from mRNA using a biotinylated oligo(dT) primer to create cDNAs having biotinylated ends.

Smaller fragments of cDNA are then be created using a restriction endonuclease, preferably one that would be expected to cleave most transcripts at least once. Preferably, a 4-base pair recognition site enzyme is used. More than one restriction endonuclease can also be used, sequentially or in tandem. The cleaved cDNA can then be isolated by binding to a capture medium using the label attached to the primer described above. For example, streptavidin beads are used to isolate the defined 3′ nucleotide sequence tag when the oligo dT primer for cDNA synthesis is biotinylated. Other capture systems (e.g. biotin/streptavidin, digoxigenin/anti-digoxigenin) can also be employed.

In one aspect, the isolated defined nucleotide sequence tags are separated into two pools of cDNA. Each pool is ligated restriction endonucleases to appropriate restriction endonucleases to linkers. The linkers can be the same or different, although when the linkers have the same sequence, it is not necessary to separate the tags into pools. The first oligonucleotide linker comprises a first sequence for hybridization of a PCR primer and the second oligonucleotide linker comprises a second sequence for hybridization of a PCR primer. In addition, the linkers further comprise a second restriction endonuclease site. The linkers are designed so that cleavage of the ligation products with the second restriction enzyme results in release of the linker having a defined nucleotide sequence tag (e.g., 3′ of the restriction endonuclease cleavage site). The defined nucleotide sequence tag may be from about 6 to 30 base pairs. Preferably, the tag is about 9 to 11 base pairs. Therefore, a ditag (i.e. the dimer of two sequence tags) is from about 12 to 60 base pairs, and preferably from 18 to 22 base pairs.

Typically, the second restriction endonuclease cleaves at a site distant from or outside of the recognition site. For example, the second restriction endonuclease can be a type IIS restriction enzyme. Type IIS restriction endonucleases cleave at a defined distance up to 20 bp away from their asymmetric recognition sites (Szybalski, W. (1985) Gene 40:169). Examples of type IIS restriction endonucleases include BsmFI and Fokl. Other similar enzymes will be known to those of skill in the art (see, CURRENT PROTOCOLS IN MOLECULAR BIOLOGY, supra).

The pool of defined tags ligated to linkers having the same sequence, or the two pools of defined nucleotide sequence tags ligated to linkers having different nucleotide sequences, are randomly ligated to each other “tail to tail”. The portion of the cDNA tag furthest from the linker is referred to as the “tail”. This creates the ditag (ligated tag pair) having a first restriction endonuclease site upstream (5′) and a first restriction endonuclease site downstream (3′) of the ditag; a second restriction endonuclease cleavage site upstream and downstream of the ditag, and a linker oligonucleotide containing both a second restriction enzyme recognition site and an amplification primer hybridization site upstream and downstream of the ditag. In other words, the ditag is flanked by the first restriction endonuclease site, the second restriction endonuclease cleavage site and the linkers, respectively.

The ditag can be amplified by utilizing primers which specifically hybridize to one strand of each linker. Preferably, the amplification is performed after the ditags have been ligated together using standard polymerase chain reaction (PCR) methods as described for example in U.S. Pat. No. 4,683,195. Alternatively, the ditags can be amplified by cloning in prokaryotic-compatible vectors or by other amplification methods known to those of skill in the art. Those of skill in the art can prepare similar primers for amplification based on the nucleotide sequence of the linkers without undue experimentation.

Cleavage of the amplified PCR product with the first restriction endonuclease allows isolation of ditags which can then be concatenated by ligation. After ligation, it may be desirable to clone the concatemers, although it is not required. Analysis of the ditags or concatemers, whether or not amplification was performed, can be performed by standard sequencing methods. Concatemers generally consist of about 2 to 200 ditags and preferably from about 8 to 20 ditags. While these are preferred concatemers, it will be apparent that the number of ditags which can be concatenated will depend on the length of the individual tags and can be readily determined by those of skill in the art without undue experimentation. After formation of concatemers, multiple tags can be cloned into a vector for sequence analysis, or alternatively, ditags or concatemers can be directly sequenced without cloning by methods known to those of skill in the art, either manually or using automated methods.

Among the standard procedures for cloning the defined nucleotide sequence tags of the invention is insertion of the tags into vectors such as plasmids or phage. The ditag or concatemers of ditags produced by the method described herein are cloned into recombinant vectors for further analysis, e.g., sequence analysis, plaque/plasmid hybridization using the tags as probes, by methods known to those of skill in the art. Vectors in which the ditags are cloned can be transferred into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

Transformation of a host cell with a vector containing ditag(s) may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed by electroporation or other commonly used methods in the art.

The individual tags or ditags, can be hybridized with oligonucleotides immobilized on a solid support (e.g., nitrocellulose filter, glass slide, silicon chip). In addition, either the ditags or Oligonucleotide probes are labeled with a detectable label, for example, with a radioisotope, a fluorescent compound, a bioluminescent compound, a chemi-luminescent compound, a metal chelator, or an enzyme. Those of ordinary skill in the art will know of other suitable labels for binding to the ditag, or will be able to ascertain such using routine experimentation. For example, PCR can be performed with labeled (e.g., fluorescein tagged) primers.

The ditags are separated into single-stranded molecules which are preferably serially diluted and added to a solid support (e.g., a silicon chip as described by Fodor et al. (1991) Science 251:767) containing oligonucleotides representing, for example, every possible permutation of a 10-mer (e.g. in each grid of a chip). The solid support is then used to determine differential expression of the tags contained within that support (e.g., on a grid on a chip) by hybridization of the oligonucleotides on the solid support with tags produced from cells under different conditions (e.g., different stage of development growth of cells in the absence and presence of a growth factor, normal versus transformed cells, comparison of different tissue expression, etc.). In the case of fluoresceinated end labeled ditags, analysis of fluorescence is indicative of hybridization to a particular 10-mer. When the immobilized oligonucleotide is fluoresceinated, for example, a loss of fluorescence due to quenching (by the proximity of the hybridized ditag to the labeled oligo) is observed and is analyzed for the pattern of gene expression.

The tag from a sequence can also be compared to a sequence database, for example using a computer method compare sequences with known sequences.

A linear search through such a database may be used. Alternatively, a sequence tag can be converted into a unique numeric representation. The tag comparison aspects may be implemented in hardware or software, or a combination of both. Preferably, these aspects of the invention are implemented in computer programs executing on a programmable computer comprising a processor, a data storage system (including volatile and non-volatile memory and/or storage elements), at least one input device, and at least one output device. Data input through one or more input devices for temporary or permanent storage in the data storage system includes sequences, and may include previously generated tags and tag codes for known and/or unknown sequences. Program code is applied to the input data to perform the functions described above and generate output information. The output information is applied to one or more output devices, in known fashion.

Each such computer program is preferably stored on a storage media or device (e.g., ROM or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

### V. Vectors Useful in Genetic Modifications

In general, genetic modifications of cells employed in the present invention are accomplished by introducing a vector containing a polypeptide or transgene encoding a heterologous or an altered antigen. A variety of different gene transfer vectors, including viral as well as non-viral systems can be used. Viral vectors useful in the genetic modifications of this invention include, but are not limited to adenovirus, adeno-associated virus vectors, retroviral vectors and adeno-retroviral chimeric vectors.

Construction of Recombinant Adenoviral Vectors or Adeno-Associated Virus Vectors. Adenovirus and adeno-associated virus vectors usefull in the genetic modifications of this invention may be produced according to methods already taught in the art. (see, e.g., Karisson et al. (1986) EMBO 5:2377; Carter (1992) Current Opinion in Biotechnology 3:533-539; Muzcyzka (1992) Current Top. Microbiol. Immunol. 158:97-129; GENE TARGETING: A PRACTICAL APPROACH (1992) ed. A. L. Joyner, Oxford University Press, NY). Several different approaches are feasible. Preferred is the helper-independent replication deficient human adenovirus system.

The recombinant adenoviral vectors based on the human adenovirus 5 (Virology 163:614-617, 1988) are missing essential early genes from the adenoviral genome (usually E1A/E1B), and are therefore unable to replicate unless grown in permissive cell lines that provide the missing gene products in trans. In place of the missing adenoviral genomic sequences, a transgene of interest can be cloned and expressed in cells infected with the replication deficient adenovirus. Although adenovirus-based gene transfer does not result in integration of the transgene into the host genome (less than 0.1% adenovirus-mediated transfections result in transgene incorporation into host DNA), and therefore is not stable, adenoviral vectors can be propagated in high titer and transfect non-replicating cells. Human 293 cells, which are human embryonic kidney cells transformed with adenovirus E1A/E1B genes, typify useful, permissive cell lines. However, other cell lines which allow replication-deficient adenoviral vectors to propagate therein can be used, including HeLa cells.

Additional references describing adenovirus vectors and other viral vectors which could be used in the methods of the present invention include the following: Horwitz, M. S., Adenoviridae and Their Replication, in Fields, B. et al. (eds.) VIROLOGY, Vol. 2, Raven Press New York, pp. 1679-1721, 1990); Graham, F. et al., pp. 109-128 in METHODS IN MOLECULAR BIOLOGY, Vol. 7: GENE TRANSFER AND EXPRESSION PROTOCOLS, Murray, E. (ed.), Humana Press, Clifton, N.J. (1991); Miller, N. et al. (1995) FASEB Journal 9:190-199 Schreier, H (1994) Pharmaceutica Acta Helvetiae 68:145-159; Schneider and French (1993) Circulation 88:1937-1942; Curiel D. T. et al. (1992) Human Gene Therapy 3: 147-154; Graham, F. L. et al., WO 95/00655 (5 Jan. 1995); Falck-Pedersen, E. S., WO 95/16772 (22 Jun. 1995); Denefle, P. et al., WO 95/23867 (8 Sep. 1995); Haddada, H. et al., WO 94/26914 (24 Nov. 1994); Perricaudet, M. et al., WO 95/02697 (26 Jan. 1995); Zhang, W. et al., WO 95/25071 (12 Oct. 1995). A variety of adenovirus plasmids are also available from commercial sources, including, e.g., Microbix Biosystems of Toronto, Ontario (see, e.g., Microbix Product Information Sheet: Plasmids for Adenovirus Vector Construction, 1996). See also, the papers by Vile et al. (1997) Nature Biotechnology 15: 840-841; Feng et al. (1997) Nature Biotechnology, 15: 866-870, describing the construction and use of adeno-retroviral chimeric vectors that can be employed for genetic modifications.

Additional references describing AAV vectors which could be used in the methods of the present invention include the following: Carter, B., HANDBOOK OF PARVOVIRUSES, Vol. I, pp. 169-228, 1990; Berns, VIROLOGY, pp. 1743-1764 (Raven Press 1990); Carter, B. (1992) Curr. Opin. Biotechnol. 3: 533-539; Muzyczka, N. (1992) Current Topics in Micro and Immunol, 158: 92-129; Flotte, T. R. et al. (1992) Am. J. Respir. Cell Mol. Biol. 7:349-356; Chatterjee et al. (1995) Ann. NY Acad. Sci. 770: 79-90; Flotte, T. R. et al., WO 95/13365 (18 May 1995); Trempe, J. P. et al., WO 95/13392 (18 May 1995); Kotin, R., Human Gene Therapy, 5: 793-801, 1994; Flotte, T. R. et al. (1995) Gene Therapy 2:357-362; Allen, J. M., WO 96/17947 (13 Jun. 1996); and Du et al. (1996) Gene Therapy 3: 254-261.

Construction of Retroviral Vectors

Retroviral vectors useful in the methods of this invention are produced recombinantly by procedures already taught in the art. For example, WO 94/29438 describes the construction of retroviral packaging plasmids and packaging cell lines. As is apparent to the skilled artisan, the retroviral vectors useful in the methods of this invention are capable of infecting the cells described herein. The techniques used to construct vectors, and transfix and infect cells are widely practiced in the art. Examples of retroviral vectors are those derived from murine, avian or primate retroviruses. Retroviral vectors based on the Moloney murine leukemia virus (MoMLV) are the most commonly used because of the availability of retroviral variants that efficiently infect human cells. Other suitable vectors include those based on the Gibbon Ape Leukemia Virus (GALV) or HIV.

The host range of retroviral vectors has been altered by substituting the env protein of the base virus with that of a second virus. The resulting, “pseudotyped”, virus has the host range of the virus donating the envelope protein and expressed by the packaging cell line. Recently, the G-glycoprotein from vesicular stomatitis virus (VSV-G) has been substituted for the MoMLV env protein. Burns et al. (1993) PNAS 90:8033-8037; and PCT patent application WO 92/14829. Since infection is not dependent on a specific receptor, VSV-G pseudotyped vectors have a broad host range.

More than one gene can be administered per vector or alternatively, more than one gene can be delivered using several compatible vectors. Depending on the genetic defect, the therapeutic gene can include the regulatory and untranslated sequences. For gene therapy in human patients, the therapeutic gene will generally be of human origin although genes from other closely related species that exhibit high homology and biologically identical or equivalent function in humans may be used, if the gene product does not induce an adverse immune reaction in the recipient. The therapeutic gene suitable for use in treatment will vary with the disease.

A marker gene can be included in the vector for the purpose of monitoring successful transduction and for selection of cells into which the DNA has been integrated, as against cells which have not integrated the DNA construct. Various marker genes include, but are not limited to, antibiotic resistance markers, such as resistance to G418 or hygromycin. Less conveniently, negative selection may be used, including, but not limited to, where the marker is the HSV-tk gene, which will make the cells sensitive to agents such as acyclovir and gancyclovir. Alternatively, selections could be accomplished by employment of a stable cell surface marker to select for transgene expressing cells by FACS sorting. The NeoR (neomycin/G418 resistance) gene is commonly used but any convenient marker gene whose sequences are not already present in the recipient cell, can be used.

The viral vector can be modified to incorporate chimeric envelope proteins or nonviral membrane proteins into retroviral particles to improve particle stability and expand the host range or to permit cell type-specific targeting during infection. The production of retroviral vectors that have altered host range is taught, for example, in WO 92/14829 and WO 93/14188. Retroviral vectors that can target specific cell types in vivo are also taught, for example, in Kasahara et al. (1994) Science 266:1373-1376. Kasahara et al. describe the construction of a Moloney leukemia virus (MoMLV) having a chimeric envelope protein consisting of human erythropoietin (EPO) fused with the viral envelope protein. This hybrid virus shows tissue tropism for human red blood progenitor cells that bear the receptor for EPO, and is therefore useful in gene therapy of sickle cell anemia and thalassemia. Retroviral vectors capable of specifically targeting infection of cells are preferred for in vivo gene therapy.

Expression of the transferred gene can be controlled in a variety of ways depending on the purpose of gene transfer and the desired effect. Thus, the introduced gene may be put under the control of a promoter that will cause the gene to be expressed constitutively, only under specific physiologic conditions, or in particular cell types.

Examples of promoters that may be used to cause expression of the introduced sequence in specific cell types include Granzyme A for expression in T-cells and NK cells, the CD34 promoter for expression in stem and progenitor cells, the CD8 promoter for expression in cytotoxic T-cells, and the CD11b promoter for expression in myeloid cells.

Inducible promoters may be used for gene expression under certain physiologic conditions. For example, an electrophile response element may be used to induce expression of a chemoresistance gene in response to electrophilic molecules. The therapeutic benefit may be further increased by targeting the gene product to the appropriate cellular location, for example the nucleus, by attaching the appropriate localizing sequences.

The vector construct is introduced into a packaging cell line which will generate infectious virions. Packaging cell lines capable of generating high titers of replication-defective recombinant viruses are known in the art, see for example, WO 94/29438. Viral particles are harvested from the cell supernatant and purified for in vivo infection using methods known in the art such as by filtration of supernatants 48 hours post transfection. The viral titer is determined by infection of a constant number of appropriate cells (depending on the retrovirus) with titrations of viral supernatants. The transduction efficiency can be assayed 48 hours later by a variety of methods, including Southern blotting.

After viral transduction, the presence of the viral vector in the transduced cells or their progeny can be verified such as by PCR. PCR can be performed to detect the marker gene or other virally transduced sequences. Generally, periodic blood samples are taken and PCR conveniently performed using e.g. NeoR probes if the NeoR gene is used as marker. The presence of virally transduced sequences in bone marrow cells or mature hematopoietic cells is evidence of successful reconstitution by the transduced cells. PCR techniques and reagents are well known in the art, See, generally, PCR PROTOCOLS, A GUIDE TO METHODS AND APPLICATIONS. Innis, Gelfand, Sninsky & White, eds. (Academic Press, Inc., San Diego, 1990) and commercially available (Perkin-Elmer).

Transduction Protocol for DCs: In vitro/ex vivo, exposure of human DCs to vector at a multiplicity of infection (MOI) of 500 for 16-24 h (e.g., Ad vector) in a minimal volume of serum-free medium reliably gives rise to transgene expression in 90-100% of DCs. The efficiency of transduction of DCs or other APCs can be assessed by immunofluorescence using fluorescent antibodies specific for the tumor antigen being expressed. Alternatively, the antibodies can be conjugated to an enzyme (e.g. HRP) giving rise to a colored product upon reaction with the substrate. The actual amount of TAA being expressed by the APCs can be evaluated by ELISA.

Transduced APCs can subsequently be administered to the host via an intravenous, subcutaneous, intranasal, intramuscular or intraperitoneal route of delivery.

In vivo'transduction of DCs, or other APCs, can potentially be accomplished by administration of Ad (or other viral vectors) via different routes including intravenous, intramuscular, intranasal, intraperitoneal or cutaneous delivery. The preferred method is cutaneous delivery of Ad vector at multiple sites using a total dose of approximately 1×10¹⁰-10¹² i.u. Levels of in vivo transduction can be roughly assessed by co-staining with antibodies directed against APC marker(s) and the TAA being expressed. The staining procedure can be carried out on biopsy samples from the site of administration or on cells from draining lymph nodes or other organs where APCs (in particular DCs) may have migrated. The amount of TAA being expressed at the site of injection or in other organs where transduced APCs may have migrated can be evaluated by ELISA on tissue homogenates.

Although viral gene delivery is more efficient, DCs can also be transduced in vitro/ex vivo by non-viral gene delivery methods such as electroporation, calcium phosphate precipitation or cationic lipid/plasmid DNA complexes. Transduced APCs can subsequently be administered to the host via an intravenous, subcutaneous, intranasal, intramuscular or intraperitoneal route of delivery.

In vivo transduction of DCs, or other APCs, can potentially be accomplished by administration of cationic lipid/plasmid DNA complexes delivered via the intravenous, intramuscular, intranasal, intraperitoneal or cutaneous route of administration. Gene gun delivery or injection of naked plasmid DNA into the skin also leads to transduction of DCs. Intramuscular delivery of plasmid DNA may also be used for immunization.

The transduction efficiency and levels of transgene expression can be assessed as described above for viral vectors.

### VI. Adoptive Immunotherapy and Vaccines

The expanded populations of antigen-specific immune effector cells of the present invention also find use in adoptive immunotherapy regimes and as vaccines.

Adoptive immunotherapy methods involve, in one aspect, administering to a subject a substantially pure population of educated, antigen-specific immune effector cells made by culturing naïve immune effector cells with APCs as described above. Preferably, the APCs are dendritic cells or dendritic cell hybrids. An effective amount of the composition is administered to induce an immune response in the subject.

In one embodiment, the adoptive immunotherapy methods described herein are autologous. In this case, the APCs are made using parental cells isolated from a single subject. The expanded population also employs T cells isolated from that subject. Finally, the expanded population of antigen-specific cells is administered to the same patient.

In another embodiment, the adoptive immunotherapy methods are allogeneic. Here, cells from two or more patients are used to generate the APCs, and stimulate production of the immune effector cells. For instance, cells from other healthy or diseased subjects can be used to generate antigen-specific cells in instances where it is not possible to obtain autologous T cells and/or dendritic cells from the subject providing the biopsy. The expanded population can be administered to any one of the subjects from whom cells were isolated, or to another subject entirely.

In a further embodiment, APCs or immune effector cells are administered with an effective amount of a stimulatory cytokine, such as IL-2 or a co-stimulatory molecule.

The agents identified herein as effective for their intended purpose can be administered to subjects or individuals susceptible to or at risk of developing a disease, such as cancer. When the agent is administered to a subject such as a mouse, a rat or a human patient, the agent can be added to a pharmaceutically acceptable carrier and systemically or topically administered to the subject. To determine patients that can be beneficially treated, a tumor regression can be assayed. Therapeutic amounts can be empirically determined and will vary with the pathology being treated, the subject being treated and the efficacy and toxicity of the therapy. When delivered to an animal, the method is useful to further confirm efficacy of the agent.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician.

The agents and compositions of the present invention can be used in the manufacture of medicaments and for the treatment of humans and other animals by administration in accordance with conventional procedures, such as an active ingredient in pharmaceutical compositions.

More particularly, an agent of the present invention also referred to herein as the active ingredient, may be administered for therapy by any suitable route including oral, rectal, nasal, topical (including transdermal, aerosol, buccal and sublingual), vaginal, parental (including subcutaneous, intramuscular, intravenous and intradermal) and pulmonary. It will also be appreciated that the preferred route will vary with the condition and age of the recipient, and the disease being treated.

### VII. Therapeutics with Identified Antigens

Once the genes and antigens have been identified, they can be employed in a variety of ways.

Cancer treatment and prevention. Cancer cells contain many new antigens potentially recognizable by the immune system. Given the speed with which these antigens can now be identified, custom anti-cancer vaccines can be generated for affected individuals by isolating TILs from patients with solid tumors, determining their MHC restriction, and assaying these CTLs against the appropriate library for reactive epitopes. The short time frame heralds a new therapeutic clinical treatment modality for cancer patients. These vaccines will be both treatments for affected individuals as well as preventive therapy against recurrence (or establishment of the disease in patients which present with a familial genetic predisposition to it). Inoculation of individuals who have never had the cancer is expected to be quite successful as preventive therapy, even though a tumor antigen-specific CTL response has not yet been elicited, because in most cases high affinity peptides seem to be immunogenic suggesting that holes in the functional T cell repertoire, if they exist, may be relatively rare (Sette et al. (1994) J. Immunol. 153:5586-5592). In mice, vaccination with appropriate antigens or epitopes not only eliminates established tumors but also protects against tumor re-establishment after inoculation with otherwise lethal doses of tumor cells (Bystryn et al. (1993) supra).

Recent advances in vaccine adjuvants provide effective means of administering peptides so that they impact maximally on the immune system (Del-Giudice, (1994) Experientia 50:1061-1066). These peptide vaccines will be of great value in treating metastatic tumors that are generally unresponsive to conventional therapies. Note that tumors arising from the homozygous deletion of recessive oncogenes are not likely to betray themselves to a humoral (antibody) response and would thus be treated more effectively by eliciting a cellular, CTL response. The ability to catalog large numbers of CTL epitopes with this technology will allow the identification of widely cross-reactive epitopes that are shared between independently derived tumors. In the case of melanoma, there is recent evidence that the same T-cell-defined tumor antigens are expressed by independent human melanoma and breast cancers suggesting that transformation-associated events may give rise to recurrent expression of the same tumor antigen in different tumors of related tissue and cellular origin (Sahasrabudhe et al. (1993) supra).

Viral Diseases. Viral infections are also ideal candidates for immunotherapy. Immunological responses to viral pathogens are sometimes ineffective as in the case of the lentiviruses such as HIV which causes AIDS. The high rates of spontaneous mutation make these viruses elusive to the immune system. However, a saturating profile of CTL epitopes presented on infected cells will identify shared antigens among different serotypes in essential genes that are largely intolerant to mutation which would allow the design of more effective vaccines.

Autoimmune Diseases. These are diseases in which the body's immune system responds against self tissues. They include most forms of arthritis, ulcerative colitis, and multiple sclerosis. This technology can identify the endogenous elements that are recognized as foreign—a giant step towards the development of treatments using gene therapy or other approaches. One of our interests is the design of synthetic CTL epitopes which can act as “suicide substrates” for CTLs that mediate autoimmunity. That is to say, peptides which have a high affinity for the MHC allele but fail to activate the TCR could effectively mask the cellular immune response against cells presenting the antigen in question. In support of this approach, it is believed that the long latency period of the HIV virus is due to an antiviral immune response and a mechanism by which the virus finally evades the immune system is by generating epitopes that occupy the MHC molecules but do not stimulate a TCR lytic response, inducing specific T cell anergy (Klenerman et al. (1995) Eur. J. Immunol. 25:1927-1931).

Diagnostic Reagents. Defined CTL epitopes can be used to clinically characterize tumors and viral pathogens in order to determine, in advance, the predicted efficacy of an in vivo vaccine trial. This can be achieved by a simple proliferation assay of a patient's peripheral blood mononuclear cells using defined CTL epitopes as stimulators. Peptides which elicit a response are viable vaccine candidates for that patient. Cataloging large numbers of CTL epitopes of defined MHC restrictions, as can be achieved with this technology, will make feasible the rapid typing and customized vaccine formulation for affected or genetically predisposed individuals.

Identification of Tumor Genes. It is predicted that optimally reactive peptides will, more often than not, reflect the structure of naturally occurring epitopes. This is because, in general, there are fewer ways to generate gain-of-function rather than loss-of-function mutations by amino acid substitutions. It is possible to clone genes which contain the defined epitope within their sequence by classical methods (i.e., hybridization of synthetic oligonucleotides to phage libraries, RT-PCR, antibody screening of phage expression libraries, etc.). Since many proteins will contain processing sites which will generate peptides that bind to a variety of MHC alleles, vaccination with the complete protein from which the natural epitope was derived will allow the design of vaccines which can largely overcome the problem of MHC restriction. It may be possible to identify these proteins based on their representation in currently available sequence databases (i.e., genbank, PIR, Swiss-Prot, etc.). Protein sequences which contain more than one identified epitope (or derivatives of identified epitopes) would be strong candidates for vaccines which may be independent of MHC restriction.

It is expected that the identification of proteins from tumor cells or vitally infected cells that are targeted by the immune system will identify genes that play a direct role in their presented abnormal phenotypes. Recently (Wolfel et al. (1995) Science 269:1281-4), an HLA-A2. 1-restricted human anti-melanoma CTL epitope which corresponds to a UV-specific mutation in the cyclin-dependent kinase 4 gene (CDK4) was identified. This is the first example of the identification of a gene responsible for tumorogenesis by isolation and analysis of an anti-tumor CTL epitope. The utilization of CTLs may be an effective means of pursuing tumor genes, possibly more effective than the conventional techniques of subtractive hybridization or representational difference analysis (Lisitsyn et al. (1993) Science 259:946-51). Knowledge of these genes will aid in understanding the molecular mechanisms underlying tumorogenesis and may suggest other clinical treatment modalities such as gene therapy.

Induction of Active Immunity Through CTL Infusion. Objective antitumor responses can be observed when TILs are infused with IL-2 in patients with metastatic melanoma (Rosenberg et al. (1991) In Biologic Therapy of Cancer, Devita et al., eds. Philidelphia: Lippincott, pp. 214-236; Rosenberg (1988) N. Engl. J. Med. 319:1676-1680). The proliferation of TILs in vitro is dependent on the persistent presence of antigen. Traditionally, the source of antigen is irradiated cells grown from the tumor from which the TILs were isolated. Most of the antitumor CTLs obtained thus far have been generated against melanomas, because metastatic melanoma cells are rather easy to adapt to culture, providing convenient sources of antigen for CTLs with unknown epitopes (Van Pel et al. (1995) Immunol. Rev. 145:229-250). It is critical to obtain antigen sources in order to stimulate CTLs to expand clones for use in CTL infusion therapy. In most cases (e.g. prostate cancer, pancreatic carcinoma, lung tumors, ovarian cancer), it is difficult to establish continuous cultures from primary explants. In these cases, the method described above is unique in that parental tumor cell line required only for an initial small-scale expansion of the CTLs and to test the efficacy of the identified peptide epitopes.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

1. A method of identifying a polynucleotide fragment of a gene that encodes an antigen recognized by an immune effector cell, comprising:
(a) providing a first cell that expresses an antigen recognized by the immune effector cell and having an identified major histocompatibility complex (MHC) restriction and one or more second cells having a compatible major histocompatibility complex (MHC) to the first cell but which does not express antigen;
(b) identifying polynucleotides encoding a peptide sequence motif in the antigen displayed by antigen presenting cells and recognized by the immune effector cell;
(c) identifying polynucleotides which are aberrantly expressed by the first cells as compared to one or more second cells; and
(d) comparing the polynucleotides identified in step (c) with the polynucleotides motifs identified in step (b) to identify the fragment of the gene encoding the antigen recognized by the immune effector cell.

2. The method of identifying a fragment of one or more genes encoding an antigen recognized by an immune effector cell, comprising:
(a) generating a first set of polynucleotide primers corresponding to peptide sequence motifs displayed by an antigen expressing cell and recognized by immune effector cells in a manner restricted by the major histocompatability complex of the antigen expressing cell;
(b) generating a second set of polynucleotide primers corresponding to the unique identifier sequences (SAGE tags) of transcripts differentially expressed in the same antigen expressing cells as described in part (a) above when compared to other antigen expressing cells that are not recognized by the same immune effector cells as in part (a) above but possess a compatible major histocompatability complex;
(c) combining the first and second sets of primers in a combinatorial fashion and identifying the transcripts representative of one or more genes encoding antigens recognized by immune effector; and
(d) amplifying cDNA obtained from the first group of cells recognized by the immune effector cells with the combined primers and analyzing the amplified products, thereby identifying one or more fragments of genes encoding antigens recognized by immune effector cells.

3. The method of claim 1, wherein step (c) comprises:
(a) providing complementary deoxyribonucleic acid (cDNA) polynucleotides from an antigen expressing cell recognized by the immune effector cells;
(b) providing cDNA polynucleotides from cells having a compatible major histocompatability complex (MHC) to the cells of step (a) but which do not express antigen;
(c) determining and analyzing the cDNAs that are aberrantly expressed by the first cells as compared to the second cells.

4. The method according to claim 1, wherein step (c) is performed before step (b).

5. The method according to claim 1 or 2, wherein the immune effector cells are cytotoxic T lymphocytes (CTLs).

6. The method according to claim 5, wherein the CTLs are selected from a group consisting of polyclonal T cells isolated from one-individual, polyclonal T cells isolated from two or more individuals sharing the same MHC restriction, two or more CTLs or any combination thereof.

7. The method according to claim 5, wherein the CTLs recognize an antigen expressed on a neoplastic cell.

8. The method according to claim 7, wherein the neoplastic cell is a tumor cell.

9. The method according to claims 1 or 2, wherein the antigen recognized by the immune effector cell is expressed on a cell site selected from the group consisting of a site of viral infection, a site of autoimmune infiltration, a site of transplantation rejection, a site of inflammation or a site of lymphocyte or leukocyte infiltration.

10. The method according to claim 1, wherein the antigen presenting cell is selected from the group consisting of a cells having a purified MHC class I molecule complexed to a β₂-microglobulin; an intact antigen presenting cell; and a foster antigen presenting cell.

11. The method according to claim 1, wherein the first cell that expresses the antigen recognized by an immune effector cell is a foster antigen presenting cell.

12. The method according to claim 1, wherein the second cell that does not express antigen is a foster antigen presenting cell that lacks antigen processing activity and expresses MHC molecules free of bound peptides.

13. The method according to claims 1 or 2, further comprising isolating the gene encoding the antigen recognized by immune effector cells.

14. A method of administering a vaccine comprising administering to a subject an effective amount of a first protein comprising a polypeptide encoded by the gene prepared by the method of claim 13.

15. A method of claims 1 or 2, further comprising inserting the gene into a suitable host cell and administering an effective amount of the transduced host cell to a subject.

16. A method of claims 1 or 2, further comprising expression cloning of the gene encoding the antigen.

17. The method of claim 13, further comprising administering an effective amount of the gene to a subject.

18. The method of claim 14, further comprising coadministering an effective amount of a cytokine.

19. The method of claim 14, further comprising coadministering an effective amount of a co-stimulatory molecule.

20. A polynucleotide fragment identified by the method of claim 1.

21. A host cell comprising the polynucleotide fragment of claim 20.

22. A vector comprising the polynucleotide fragment of claim 20.

23. A gene comprising the polynucleotide fragment of claim 1.

24. A polynucleotide encoded by the polynucleotide fragment of claim 20.
